(19) United States
(12) Patent Application Publication
(10) Pub. No.: US 2024/0418704 A1
(43) Pub. Date: Dec. 19, 2024

(54) EPIGENETIC TARGETS IN CLONAL HEMATOPOIESIS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ross Levine, New York, NY (US); Robert Lyle Bowman, Wynnewood, PA (US); Michael Robert Waarts, New York, NY (US)

(73) Assignees: Memorial Sloan-Kettering Cancer Center, New York, NY (US); Memorial Hospital for Cancer and Allied Diseases, New York, NY (US); Sloan-Kettering Institute for Cancer Research, New York, NY (US)

(21) Appl. No.: 18/706,831

(22) PCT Filed: Nov. 3, 2022
(86) PCT No.: PCT/US2022/048816

§ 371 Date: May 2, 2024

Related U.S. Application Data
(60) Provisional application No. 63/275,306, filed on Nov. 3, 2021.

(51) Int. Cl. G01N 33/50; C12N 5/0789; C12N 9/22; C12N 15/10; C12N 15/11

(52) U.S. Cl. CPC G01N 33/5023; C12N 5/0647; C12N 9/22; C12N 15/1058; C12N 15/111; G01N 33/5073; C12N 2310/20; C12N 2502/13

(57) ABSTRACT

The present disclosure provides, in some embodiments, methods for culturing and expanding hematopoietic stem cells (HSPCs) comprising a genomic modification associated with clonal hematopoiesis (CH). These cultured and expanded HSPCs are used, in some embodiments, to identify genes that promote CH, to identify inhibitors of CH, and to inhibit CH.

### CROSS-REFERENCE TO RELATED APPLICATIONS

This application claims priority under 35 U.S.C. § 119(e) to U.S. Provisional Patent Application No. 63/275,306, filed Nov. 3, 2021, which is hereby incorporated by reference in its entirety.

### GOVERNMENT SUPPORT

This invention was made with government support under CA173636 and CA197594, awarded by the National Institutes of Health. The government has certain rights in this invention.

### BACKGROUND

Clonal hematopoiesis (CH) is defined as the outgrowth of a genetically distinct subpopulation of cells in the blood and is common with aging, affecting greater than 10% of the population over 65 years of age. CH is associated with an increased risk of hematological malignancy and all-cause mortality, due at least in part to an increased risk of cardiovascular disease. Eliminating or mitigating the expansion of CH-mutant cells is thus of potential therapeutic interest in both preventing transformation to hematological malignancies and mitigating cardiovascular disease and other-aging related diseases.

### SUMMARY

The present disclosure is based on the development of compositions and methods for culturing and expanding hematopoietic stem cells (HSPCs) comprising a genomic modification associated with CH ex vivo. Unexpectedly, these cultured and expanded HSPCs recapitulate a CH phenotype rapidly (e.g., over 1-2 weeks) compared with previous in vivo studies. Additionally, cultured HSPCs comprising a genomic modification associated with CH that are transplanted into a subject recapitulate in vivo CH phenotypes. Thus, methods and cell cultures provided herein may be used to identify genes that promote CH, to identify inhibitors of CH, and to inhibit CH.

In some aspects, the present disclosure provides a cell culture comprising HSPCs and endothelial cells, wherein the HSPCs comprise: a first HSPC comprising a genomic modification associated with CH; and a second HSPC that does not comprise the genomic modification associated with CH.

In some aspects, the present disclosure provides a method for expanding HSPCs, the method comprising culturing HSPCs with endothelial cells. In some embodiments, the HSPCs comprise a genomic modification associated with CH.

In some aspects, the present disclosure provides a method of identifying a gene that promotes CH, the method comprising: contacting HSPCs with an agent that modulates expression of a gene in the HSPCs, wherein the HSPCs comprise a genomic modification associated with CH; culturing the contacted HSPCs with endothelial cells; and determining an expression level of the gene in the cultured HSPCs. In some embodiments, a decrease in the expression level relative to a control indicates that the gene promotes CH.

In some aspects, the present disclosure provides a method of identifying an inhibitor of CH, the method comprising: contacting HSPCs with a test compound, wherein the HSPCs comprise a genomic modification associated with CH; culturing the contacted HSPCs with endothelial cells; and measuring survival or proliferation of the cultured HSPCs contacted with the test compound. In some embodiments, a decrease in the survival or proliferation compared to a control indicates that the test compound is an inhibitor of CH.

In some aspects, the present disclosure further provides a method of inhibiting CH, the method comprising: contacting a HSPC, the HSPC comprising a genomic modification associated with CH, with an agent that decreases the activity of a product of a gene encoding a histone 3, lysine 9 (H3K9) demethylase. In some embodiments, an agent is a genome-editing agent (e.g., sgRNA) that targets the gene encoding the H3K9 demethylase. In some embodiments, the product of the gene encoding the H3K9 demethylase is a protein. In some embodiments, an agent is an antisense oligonucleotide that targets an mRNA encoding the protein. In some embodiments, an agent is an inhibitor of the protein. In some embodiments, the inhibitor of the protein is an enzymatic inhibitor that decreases H3K9 demethylase activity of the protein. In some embodiments, the inhibitor of the protein is a mediator of protein degradation that mediates targeted degradation of the protein (e.g., H3K9 demethylase).

In some embodiments, the endothelial cells are bone marrow endothelial cells (BMECs). In some embodiments, the genomic modification comprises a substitution, insertion, or deletion in a gene encoding a protein, and the genomic modification results in expression of a variant of the protein. In some embodiments, the variant of the protein is associated with CH. In some embodiments, the genomic modification is a substitution, insertion, or deletion in a gene encoding an IDH2 protein, a TET2 protein, an ASXL1 protein, and/or a DNMT3a protein. In some embodiments, the control comprises an expression level of a gene in wild-type HSPCs.

In some embodiments, the first HSPC comprises a marker, and the second HSPC does not comprise the marker. In some embodiments, the marker is a fluorescent marker (e.g., a fluorescent protein). In some embodiments, the first HSPC comprises a genomic modification associated with CH, and the second HSPC does not comprise the genomic modification associated with CH.

In some embodiments, the method further comprises, prior to the culturing, infecting the HSPCs with a library of single guide RNAs (sgRNAs). In some embodiments, the library comprises 10-10,000 sgRNAs.

In some embodiments, the HSPCs express a programmable nuclease protein. In some embodiments, the programmable nuclease protein is Cas9.

In some embodiments, the H3K9 demethylase is KDM3B and/or JMJD1C.

The details of certain embodiments of the invention are set forth in the Detailed Description, as described below. Other features, objects, and advantages of the invention will be apparent from the Examples, Drawings, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1. A bone marrow epithelial cell (BMEC) assay for characterizing clonal hematopoiesis (CH) alleles. Long-term hematopoietic stem cells (LT-HSCs) are isolated from wild-type (WT) protein and TdTomato+mutant protein (Tet2/Dnmt3a/Asxl1/IDH2) mice and co-cultured ex vivo for 1-4 weeks with BMECs. After expanding, perturbing, and evaluating competition, wild-type and mutant cells are transplanted back into a mouse and the chimerism of cells expressing wild-type and mutant protein is evaluated. BMT is bone marrow thymus transplantation.

FIGS. 2A-2D. Ex vivo culture of cells expressing CH alleles with BMECs recapitulates in vivo studies. Lineage positive (FIG. 2A), lineage negative (FIG. 2B), Lin Sca-1⁺c-Kit⁺ (LSKs) (FIG. 2C), and LT-HSCs (FIG. 2D) cells are from wild-type (Dnmt3a, Tet2, Asxl1, Idh2), and mutant (Dnmt3a KO, Tet2 KO, Asxl1 KO, Idh2 R140Q). N.S. is not significant.

FIGS. 3A-3E. Ex vivo culture of cells expressing CH alleles with BMECs coupled with in vivo readouts demonstrate faithful recapitulation of prior murine and human data. Percent (%) of total chimeric cells expressing mutant Asxl1 (FIG. 3A), Tet2 (FIG. 3B), Dnmt3a (FIG. 3C), and Idh2 (FIG. 3D) proteins are evaluated for CD45 positive cells, CD45 positive cells not expressing TdTomato marker (CD45.2 TdT⁻), and CD45 positive cells expressing TdTomato marker (CD45.2 TdT⁺) after ex vivo culture and in vivo transplantation. FIG. 3E shows the ratio of cells expressing mutant protein/WT protein (Asxl1, Tet2, Dnmt3A, IDH2) after ex vivo culture and in vivo transplantation.

FIGS. 4A-4C. BMEC co-culture system can be used to readout cell-type specific dependencies in response to small molecules and single guide RNAs (sgRNAs). The percentage of heme-expression and cell differentiation in myeloid-derived suppressor cells (Cd11b⁺Gr1⁺), monocyte & macrophage (Cd11b⁺Gr1⁻) cells, lineage negative (Lin neg) cells, granulocyte/macrophage progenitor (GMP) cells, megakaryocyte/erythrocyte progenitors (MEPs) cells, and LSK cells was evaluated after contacting the cells with an agent that induces differentiation (Retinoic Acid, FIG. 4A), an agent that has no effect on differentiation (Venetoclax, FIG. 4B), and an agent that inhibits differentiation (Dasatanib, FIG. 4C).

FIG. 5. BMEC co-culture system as a platform for ex vivo targeted clustered regularly interspaced programmable repeats (CRISPR).

FIGS. 6A-6B. CRISPR screen of WT hematopoietic stem cells (HSPCs) identifies genetic dependencies in normal hematopoiesis. Fold change in gene expression comparing day 7 and day 1 after infecting HSPCs with an epigenome library in the absence of (FIG. 6A) and the presence of (FIG. 6B) Cas9 protein.

FIG. 7. Enriched and depleted genes in normal hematopoiesis. Fold change in gene expression comparing day 7 and day 1 after infecting HSPCs with an epigenome library in the presence of Cas9 protein. Enriched genes appear at or above the upper dashed line and Depleted genes appear at or below the lower dashed line.

FIG. 8. Wild-type HSPC gene dependencies are reproducible. The agreement between the number of read counts (log₁₀) in Replicate 1 and Replicate 2 is plotted. The Pearson correlation coefficient between the read counts in Replicate 1 and Replicate 2 is 0.969.

FIGS. 9A-9C. Mutant-specific gene dependencies in IDH2/Tet2/Asxl1 cells in bulk culture. Fold change comparing gene expression in mutant IDH2 cells (FIG. 9A, R140Q mutation), mutant Tet2 cells (FIG. 9B, Tet2 KO), and mutant Asxl1 cells (FIG. 9C, Asxl1 KO) versus wild-type cells 7 days after infecting HSPCs with an epigenome library in the presence of Cas9 protein.

FIGS. 10A-10C. Mutant-specific gene dependencies in IDH2/Tet2/Asxl1 lineage negative (lin neg) HSPCs. Fold change comparing gene expression in mutant IDH2 cells (FIG. 10A, R140Q mutation), mutant Tet2 cells (FIG. 10B, Tet2 KO), and mutant Asxl1 cells (FIG. 10C, Asxl1 KO) versus wild-type cells 14 days after infecting HSPCs with an epigenome library in the presence of Cas9 protein.

FIGS. 11A-11D. Validation of mutant-specific gene dependencies in IDH2/Tet2/Asxl1 cells in single guide RNA (sgRNA) studies. FIG. 11A shows a schematic of sgRNA studies in a WT mouse expressing Cas9 and a clonal hematopoiesis (CH) mutant mouse expressing Cas9 and TdTomato (TdT+)-CH mutant protein. FIGS. 11B-11D show the percentage of lineage negative TdT-expressing IDH2 mutant cells (FIG. 11B), Tet2 cells (FIG. 11C), and Asxl1 cells (FIG. 11D) cells 14 days after infection with sgRNAs complementary to genes identified as being mutant-specific in FIGS. 10B-10D. n=2 replicates.

FIGS. 12A-12C. Characterizing Jmjd1c and Kdm3b as candidate mutant-specific gene dependencies in Tet2^{KO}/IDH2^{R140Q}. FIG. 12A shows the percentage of lineage positive, lineage negative, LSK, GMP, and MEP TdT-expressing Tet2 mutant cells (TET2 KO) 14 days after infection with sgRNAs complementary to Rosa, Jmjd1c, and Kdm3b genes. FIGS. 12B and 12C show fold change comparing gene expression in mutant Tet2 cells (Tet2^{KO}) and mutant IDH2 cells (IDH2^{R140Q}) versus wild-type cells 7 days (FIG. 12B) and 14 days in lineage negative cells (FIG. 12C, lin neg) after infecting HSPCs with an epigenome library in the presence of Cas9 protein.

FIGS. 13A-13D. In vivo murine validation of Jmjd1c and Kdm3b as candidate mutant-specific gene dependencies in Tet2^{KO}/IDH2^{R140Q}. FIG. 13A shows the percentage of TdT-expressing Idh2 mutant cells (IDH2 R140Q) versus wild-type cells from 4-20 weeks measured in the peripheral blood of recipient mice. Donor cells from wildtype versus IDH2 R140Q mice were mixed equally and infected with a control Rosa, Kdm3b, or Jmjd1c sgRNA and chimerism was tracked in vivo. FIG. 13B shows chimerism within the bone marrow of recipient mice depicted in FIG. 13A. Chimerism is shown within all hematopoietic cells, lineage negative cells, GMPs, MEPs, LSKs, and LTHSCs. FIG. 13C shows the percentage of TdT-expressing Tet2 KO cells versus wild-type cells from 4-20 weeks measured in the peripheral blood of recipient mice. Donor cells from wildtype versus Tet2 KO mice were mixed equally and infected with a control Rosa, Kdm3b, or Jmjd1c sgRNA and chimerism was tracked in vivo. FIG. 13D shows chimerism within the bone marrow of recipient mice depicted in FIG. 13C. Chimerism is shown within all hematopoietic cells, lineage negative cells, GMPs, MEPs, LSKs, and LTHSCs.

### DETAILED DESCRIPTION

The present disclosure provides, in some aspects, a platform for studying clonal hematopoiesis (CH). This platform utilizes hematopoietic stem cells (HSPCs) comprising a genomic modification associated with CH. In some embodiments, these HSPCs are co-cultured ex vivo with bone marrow epithelial cells to allow maintenance and expansion of the CH-mutated HSPCs. In some embodiments, these HSPCs are co-cultured ex vivo with wild-type HSPCs and bone marrow endothelial cells (BMECs) to allow maintenance and expansion of the CH-mutated and wild-type HSPCs. Unexpectedly, these co-cultured CH-mutated HSPCs develop phenotypes ex vivo over a period of 7-14 days that recapitulate phenotypes which take months to develop in vivo. The mutated HSPCs may then be transplanted into a subject, where they develop CH phenotypes in vivo. Thus, the present disclosure provides a robust platform to study genetically-defined HSPCs with genomic modifications associated with CH.

This robust platform may be used, in some embodiments, to expand HSPCs comprising a genomic modification associated with CH ex vivo, to identify genes that promote CH, and to identify inhibitors of CH. Indeed, the robust platform for studying genetically-defined HSPCs with genomic modifications associated with CH was utilized to identify the histone 3, lysine 9 (H3K9) demethylase genes KDM3B and JMJD1C that promote CH. Accordingly, in some aspects, the disclosure relates to the discovery of therapeutic targets for inhibiting or treating CH.

### Clonal Hematopoiesis

Clonal hematopoiesis (CH) occurs when a hematopoietic stem cell (HSPC) has a unique genomic modification that provides a competitive advantage over HSPCs that do not have the unique genomic modification (e.g., wild-type HSPCs). A competitive advantage may be increased proliferation, increased survival, and/or increased differentiation of an HSPC having a genomic modification associated with CH compared with an HSPC that does not have the genomic modification associated with CH (e.g., a wild-type HSPC). HSPCs with the unique genomic modification differentiate into blood cells containing the same unique genomic modification as the undifferentiated HSPC.

An HSPC is a blood stem cell that is undifferentiated, proliferates indefinitely, and may mature into any of multiple mature hematopoietic cells. Non-limiting examples of mature hematopoietic cells include: common myeloid progenitor cells, megakaryotes, thrombocytes, erythrocytes, mast cells, myeloblasts, basophils, neutrophils, eosinophils, monocytes, macrophages, common lymphoid progenitor cells, natural killer cells, dendritic cells, small lymphocytes, T cells, B cells, and plasma cells. It should be understood that an HSPC having a genomic modification associated with CH also encompasses any mature hematopoietic cells that differentiate from the HSPC and also have the genomic modification associated with CH.

There is no single cause of CH, but some characteristics that increase the risk of developing CH include, but are not limited to: age, where less than 1% of the population under age 40 has CH and approximately 10-20% of the population over age 70 has CH; being male; being white; and smoking. In some instances, CH can lead to blood cancers, including myelodysplastic syndrome and acute myeloid leukemia (AML). Subjects having CH also have a higher risk of cardiovascular disease, including heart attacks.

Numerous genomic modifications are associated with CH. Genomic modifications associated with CH are unique genomic modifications that occur in CH cells, whether these CH cells are undifferentiated HSPCs or blood cells differentiated from these HSPCs. Many of these genomic modifications occur in epigenetic regulator proteins (e.g., DNMT3A, TET2, ASXL1), metabolic proteins (e.g., IDH2), signaling proteins (e.g., JAK2, CBL, GNAS), spliceosome component proteins, (e.g., SFB1, SRSF2), DNA damage response proteins (e.g., TP53, PPM1D), and apoptosis proteins (e.g., GNB1). In some embodiments, genomic modifications associated with CH occur in a gene encoding TET2, ASXL1, IDH2, DNMT3A, or some combination thereof.

In some embodiments, a genomic modification is a substitution, insertion, or deletion in a gene encoding a protein, and the genomic modification results in expression of a variant of the protein, where the variant of the protein is associated with CH. In some embodiments, the protein is an enzyme, and the genomic modification results in expression of a variant of the enzyme, where enzymatic activity of the variant of the enzyme is associated with CH. In some embodiments, the genomic modification is a substitution, insertion, or deletion in a gene encoding an IDH2 protein, a TET2 protein, an ASXL1 protein, and/or a DNMT3A protein.

In some embodiments, genomic modifications associated with CH occur in a TET2 gene. Tet methylcytosine dioxygenase 2 (TET2) encodes a protein that catalyzes the conversion of methylcytosine to 5-hydroxymethylcytosine in myelopoiesis. Myelopoiesis is the process in which innate immune cells, such as neutrophils, dendritic cells, and monocytes, develop from a myeloid progenitor cell.

TET2 is conserved in human (Gene ID: 54790), mouse (Gene ID: 214133), rat (Gene ID: 310859), zebrafish (Gene ID: 101887161), non-human primate (Gene IDs: 100407516, 116481647), chicken (Gene ID: 422540), and dog (Gene ID: 478499).

In some embodiments, TET2 is human TET2 encoding a human TET2 protein. There are 3 isoforms of human TET2 protein due to alternative splicing of the mRNA. In some embodiments, a TET2 protein is human TET2 isoform 1 (Uniprot ID: Q6N021-1), which contains amino acids 1-2,002. In some embodiments, a TET2 protein is human TET2 isoform 2 (Uniprot ID: Q6N021-2), which differs in sequence at amino acids 1137-1165 and is missing amino acids 1166-2002 with respect to human TET2 isoform 1. In some embodiments, a TET2 protein is human TET2 isoform 3 (Uniprot ID: Q6N021-3), which differs in sequence at amino acids 1137-1194 and is missing amino acids 1195-2002 with respect to human TET2 isoform 1.

Genomic modifications in TET2 are associated with CH, myelodysplastic syndrome, and polycythemia vera. Non-limiting examples of mutations in a TET2 protein that occur as a result of genomic modifications in TET2 include: TET2 knock-out (KO), R1261G, R1262A, S1290A, WSMYYN1291-1296GGSGGS, MY1293-1294AA, Y1295A, S1303N, H1382Y, D1384A, D1384V, N1387A, Y1902A, and H1904R. These amino acid residues and numbers are with respect to human TET2 protein.

In some embodiments, genomic modifications associated with CH occur in an ASXL1 gene. ASXL transcriptional regulator 1 (ASXL1) encodes a protein in the Polycomb group of proteins that maintain stable repression of genetic loci. ASXL1 protein disrupts chromatin in localized areas, enhancing transcription of certain genes and repressing the transcription of other genes.

ASXL1 is conserved in human (Gene ID: 171023), mouse (Gene ID: 228790), rat (Gene ID: 311553), zebrafish (Gene ID: 403066), non-human primate (Gene IDs: 711799, 100389269), chicken (Gene ID: 428158), and dog (Gene ID: 100688017).

In some embodiments, ASXL1 is human ASXL1 encoding a human ASXL1 protein. Human ASXL1 protein is 85 amino acids long (UniProt ID: Q498B9-1).

Genomic modifications in ASXL1 are associated with CH, myelodysplastic syndrome, and chronic myelomonocytic leukemia. Non-limiting examples of mutations in an ASXL1 protein that occur as a result of genomic modifications in ASXL1 include: an ASXL1 knock-out (KO), ASXL1^{aa-587}, Gly646TrpfsX12, Leu762PhefsX12, Trp796GlyfsX22, G1397S, G1058V, and A1312V. These amino acid residues and numbers are with respect to human ASXL1 protein.

In some embodiments, a genomic modification associated with CH occurs in an IDH2 gene. Isocitrate dehydrogenase (NADP(+)) 2 (IDH2) encodes an enzyme that catalyzes the oxidative decarboxylation of isocitrate to 2-oxoglutarate in the tricarboxylic acid (TCA) cycle in the mitochondria. IDH2 protein plays a role in intermediary metabolism and energy production.

IDH2 is conserved in human (Gene ID: 3418), mouse (Gene ID: 269951), rat (Gene ID: 361596), zebrafish (Gene ID: 386951), non-human primate (Gene IDs: 701480, 453645), chicken (Gene ID: 431056), dog (Gene ID: 479043), and pig (Gene ID: 397603).

In some embodiments, IDH2 is human IDH2 encoding a human IDH2 protein. There are 2 isoforms of human IDH2 protein due to alternative splicing of the mRNA. In some embodiments, an IDH2 protein is human IDH2 isoform 1 (Uniprot ID: P48735-1), which contains amino acids 1-452. In some embodiments, an IDH2 protein is human IDH2 isoform 2 (Uniprot ID: P48735-2), which is missing amino acid residues 1-52 with respect to human IDH2 isoform 1.

Genomic modifications in IDH2 are associated with CH, D-2-hydroxyglutaric aciduria (D2HGA2), and glioma. Non-limiting examples of mutations in an IDH2 protein that occur as a result of IDH2 genomic modifications include: R140Q, an IDH2 knock-out (KO), R140G, P158L, P162S, R172G, R172K, R172M, K413A, K413Q, and K413R. These amino acid residues and numbers are with respect to human IDH2 protein.

In some embodiments, genomic modifications (e.g., mutations) associated with CH occur in a DNMT3a gene. DNA methyltransferase 3 alpha (DNMT3a) encodes an enzyme that catalyzes de novo DNA methylation in mammalian cells. This de novo DNA methylation enables key epigenetic modifications essential for processes such as cellular differentiation, embryonic development, transcriptional regulation, heterochromatin formation, X-inactivation, imprinting, and genome stability.

DNMT3a is conserved in human (Gene ID: 1788), mouse (Gene ID: 13435), rat (Gene ID: 444984), non-human primate (Gene IDs: 694822, 739139, 100413373), chicken (Gene ID: 421991), dog (Gene ID: 482996), and pig (Gene ID: 100037301).

In some embodiments, DNMT3a is human DNMT3a encoding a human DNMT3a protein. There are 3 isoforms of human DNMT3a protein due to alternative splicing of the mRNA. In some embodiments, a DNMT3a protein is human DNMT3a isoform 1 (Uniprot ID: Q9Y6K-1), which contains amino acids 1-912. In some embodiments, a DNMT3a protein is human DNMT3a isoform 2 (Uniprot ID: Q9Y6K-2), which differs in sequence at amino acids 1-213 with respect to human DNMT3a isoform 1. In some embodiments, a DNMT3a protein is human DNMT3a isoform 3 (Uniprot ID: Q9Y6K-3), which differs in sequence at amino acids 151-166 and is missing amino acids 167-912 with respect to human DNMT3a isoform 1.

Genomic modifications in DNMT3a are associated with CH, Tatton-Brown-Rahman syndrome (TBRS), acute myelogenous leukemia (AML), and Heyn-Sproul-Jackson syndrome (HESJAS). Non-limiting examples of mutations in a DNMT3a protein that occur as a result of genomic modifications in DNMT3a include: DNMT3a knock-out (KO), I310N, Y365C, D529N, G532S, M428K, C549R, L648P, P700L, R749C, R771Q, V778G, N838D, R882C, R882H, F902S, P904L. These amino acid residues and numbers are with respect to human DNMT3a protein.

CH may occur with a combination of 1-20, 2-19, 3-18, 4-17, 5-16, 6-15, 7-14, 8-13, 9-12, or 10-11 different unique genomic modifications. In some embodiments, CH occurs with a combination of 1, 2, 3, 4, 5, 6, 7, 8, 9, 10, 11, 12, 13, 14, 15, 16, 17, 18, 19, or 20 or more different unique genomic modifications. In some embodiments, a combination of different CH genomic modifications occurs in 1-20, 2-19, 3-18, 4-17, 5-16, 6-15, 7-14, 8-13, 9-12, or 10-11 different genes. In some embodiments, a combination of different CH genomic modifications occurs in 1, 2, 3, 4, 5, 6, 7, 8, 9, 10, 11, 12, 13, 14, 15, 16, 17, 18, 19, or 20 or more different genes.

### Cell Culture

In some aspects, the present disclosure provides a cell culture comprising HSPCs and endothelial cells, where the HSPCs include HSPCs having a genomic modification associated with CH. In some embodiments, a cell culture comprises the HSPCs and the endothelial cells maintained in the same culture vessel. HSPCs having a genomic modification associated with CH have genomic modifications in any genes associated with CH provided herein or any genes otherwise known to be, or suspected of being, associated with CH. It should be understood that references to an HSPC having a genomic modification associated with CH in cell culture may also include any cells that differentiate from the HSPC and have the genomic modification associated with CH.

HSPCs may be any HSPCs known in the art. Non-limiting examples of HSPCs include: lineage negative, Lin⁻ Sca-1⁺ c-Kit⁺ (LSK), long-term HSPCs (LT-HSPC), and short-term HSPCs (ST-HSPCs).

HSPCs may be isolated from any subject that develops CH. In some embodiments, a subject is a vertebrate. A vertebrate may be any vertebrate known in the art including, but not limited to: a human, a rodent (e.g., mouse, rat, hamster), a non-human primate (e.g., Rhesus monkey, chimpanzee, orangutan), a pet (e.g., dog, cat, ferret), a livestock animal (e.g., pig, cow, sheep, chicken), or a fish (zebrafish, catfish, perch). In some embodiments, a subject from which HSPCs are isolated is a mouse.

In some embodiments, a genomic modification is a substitution, insertion, or deletion in a gene encoding a protein, and the genomic modification results in expression of a variant of the protein, where the variant of the protein is associated with CH. In some embodiments, the protein is an enzyme, and the genomic modification results in expression of a variant of the enzyme, where enzymatic activity of the variant of the enzyme is associated with CH. In some embodiments, the genomic modification is a substitution, insertion, or deletion in a gene or more than one gene encoding an IDH2 protein, a TET2 protein, an ASXL1 protein, and/or a DNMT3A protein.

Endothelial cells in cell culture with HSPCs having a genomic modification associated with CH may be any endothelial cells known in the art. Non-limiting examples of endothelial cells are bone marrow endothelial cells (BMECs), arterial endothelial cells, venous endothelial cells, endocardial endothelial cells, glomerular endothelial cells, lymphatic endothelial cells, human umbilical vein endothelial cells, microvascular endothelial cells, dermal endothelial cells, lung endothelial cells, peritubular endothelial cells, and blood brain barrier endothelial cells. In some embodiments, a cell culture comprises BMECs.

HSPCs having a genomic modification associated with CH and BMECs may be cultured with any conditions known in the art that allows survival and proliferation of HSPCs. In some embodiments, HSPCs having a genomic modification associated with CH and BMECs are cultured with the conditions described in Poulos et al., (2015), Stem Cell Reports 10; 5(5):881-894.

In some embodiments, a cell culture comprises (i) a first HSPC comprising a genomic modification associated with CH, and (ii) and a second HSPC that does not comprise the genomic modification associated with CH. As described above, HSPCs having a genomic modification associated with CH have a competitive advantage over HSPCs that do not have a genomic modification associated with CH (e.g., wild-type HSPCs). Thus, in some embodiments, the first HSPC has a competitive advantage in cell culture over the second HSPC. A competitive advantage may be any competitive advantage provided herein (e.g., increased proliferation, increased survival, increased differentiation into mature hematopoietic cells).

It should be understood that references to a first HSPC having a genomic modification associated with CH in cell culture may also include any cells that differentiate from the first HSPC and have the genomic modification associated with CH, and references to a second HSPC not having a genomic modification associated with CH in cell culture may also include any cells that differentiate from the second HSPC and do not have a genomic modification associated with CH.

It may be advantageous to easily distinguish a first HSPC having a genomic modification associated with CH from a second HSPC that does not have a genomic modification associated with CH. For example, a first HSPC may be separated from a second HSPC before engrafting the first HSPC into a subject (e.g., a mouse), before sequencing the first HSPC and measuring gene expression, or a combination thereof. A first HSPC may be distinguished from a second HSPC by any method known in the art including, but not limited to: expression of a marker, radiolabeling, and/or sequencing for the genomic modification associated with CH.

In some embodiments, a first HSPC comprises a marker that distinguishes the first HSPC from the second HSPC. In some embodiments, a second HSPC comprises a marker that distinguishes the second HSPC from the first HSPC. In some embodiments, a marker is a protein that is expressed in an HSPC or on an HSPC such that the HSPC can be distinguished from a cell that does not comprise a marker (e.g., a second HSPC). In some embodiments, a marker is a fluorescent marker, such as a fluorescent protein. Non-limiting examples of fluorescent markers include: TdTomato, GFP, EGFP, Emerald, ZsGreen, T-Sapphire, EBFP, EBFP2, Azurite, mTagBFP, ECFP, Cerulean, mTurqouise, CyPet, AmCyan1, TagCFP, YFP, Topaz, Venus, mCitrine, YPet, TagYFP, PhiYFP, mBanana, mOrange, Kusabira Orange, TagRFP, DsRed, mTangerine, mRuby, mApple, mStrawberry, RFP, JRed, mCherry, mRaspberry, and mPlum.

A cell culture comprising an HSPC (e.g., a first HSPC, a second HSPC) and endothelial cells may be maintained ex vivo for 3 days-8 weeks, 6 days-6 weeks, 1 week-5 weeks, or 2 weeks-4 weeks. In some embodiments, a cell culture is maintained for 3 days, 4 days, 5 days, 6 days, 1 week, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, 8 weeks, or longer.

### Methods for Culturing and Expanding HSPCs

Primary, genetically defined HSPCs having a genomic modification associated with CH had not been cultured ex vivo prior to the present disclosure. Thus, it was not previously possible to study CH in HSPCs by evaluating proliferation, differentiation, and response of HSPCs having a genomic modification associated with CH to external perturbations.

Accordingly, in some aspects, the present disclosure provides a method for studying CH in vitro by providing a method for expanding HSPCs having a genomic modification associated with CH, the method comprising culturing HSPCs with endothelial cells. Expanding HSPCs means that HSPCs survive and proliferate in vitro compared to HSPCs that are not cultured in vitro (e.g., with endothelial cells). Proliferate may be production of additional HSPCs having a genomic modification associated with CH or differentiation of an HSPC having a genomic modification associated with CH into a differentiated (e.g., mature) hematopoietic cell.

In some embodiments, HSPCs (e.g., having a genomic modification associated with CH, not having a genomic modification associated with CH) are primary cells isolated from a subject. An HSPC may be isolated by any method known in the art. Non-limiting methods of isolating an HSPC include: fluorescence-activated cell sorting (FACS), magnet-activated cell sorting (MACS), pre-plating, conditioned expansion media, density gradient centrifugation, field flow fractionation (FFF), and dielectrophoresis (DEP).

Endothelial cells cultured with HSPCs may be any endothelial cells provided herein. In some embodiments, HSPCs having a genomic modification associated with CH are cultured with bone marrow endothelial cells (BMECs). A genomic modification associated with CH may be any genomic modification associated with CH provided herein. In some embodiments, a genomic modification associated with CH is a substitution, insertion, or deletion in a gene encoding a protein, and the genomic modification results in expression of a variant of the protein that is associated with CH. In some embodiments, a genomic modification is a substitution, insertion, or deletion in a gene or more than one gene encoding an IDH2 protein, a TET2 protein, an ASXL1 protein, and/or a DNMT3A protein.

In some embodiments, HSPCs cultured with endothelial cells include a first HSPC having a genomic modification associated with CH and a second HSPC that does not have a genomic modification associated with CH. Thus, the first HSPC will have a competitive advantage over the second HSPC in vitro. The first HSPC may be distinguished from the second HSPC by any method known in the art or provided herein. In some embodiments, the first HSPC comprises a marker that distinguishes the first HSPC from the second HSPC (e.g., the second HSPC does not comprise the marker). In some embodiments, the second HSPC comprises a marker that distinguishes the second HSPC from the first HSPC (e.g., the first HSPC does not comprise the marker). In some embodiments, a marker is a fluorescent marker. For example, in some embodiments, the marker is a fluorescent protein, as described herein.

In some embodiments, prior to culturing, an HSPC (e.g., a first HSPC, a second HSPC) is contacted with an agent. An agent is a compound that alters the expression of one or more genes in an HSPC (e.g., a first HSPC, a second HSPC). Thus, the effect of an agent on CH may be studied by a method provided herein by contacting an HSPC with an agent prior to the culturing and studying the effect of the agent on HSPC proliferation, HSPC survival, or some combination thereof.

HSPC proliferation may be measured by any method known in the art. Non-limiting methods of measuring HSPC proliferation include: metabolic activity assays (e.g., MTT, XTT, MTS, WST1), cell proliferation marker assays (e.g., Ki-67, PCNA, topoisomerase IIB, phosphorylated histone H3), ATP concentration assays (e.g., luciferase), DNA synthesis assays (e.g., BrdU, 3H-thymine), and cell movement assays (e.g., scratch assay, agarose drop assay, cell culture insert).

HSPC survival may be measured by any method known in the art. Non-limiting methods of measuring HSPC proliferation include: colony formation assays, luciferase viability assays, ATP viability assays, tetrazolium reduction viability assays, resazurin reduction viability assays, protease release cytotoxicity assays, lactate dehydrogenase release cytotoxicity assays, and DNA dye cytotoxicity assays.

HSPC differentiation into mature hematopoietic cells may be measured by any method known in the art. Non-limiting methods of measuring HSPC differentiation include: FACS (e.g., SCA-1⁺, c-Kit⁺), MACS, Rhodamine-123 staining, and Hoescht 33342 staining.

In some embodiments, an HSPC (e.g., a first HSPC, a second HSPC) having a genomic modification associated with CH is contacted with an agent that modulates expression of a gene in the HSPC. An agent that modulates expression of a gene may be an agent that increases expression of a gene or an agent that decreases expression of a gene relative to a control. A control may be an HSPC having a genomic modification associated with CH that is not contacted with the agent or a wild-type HSPC.

Increased expression of a gene may be expression that is increased by 5%-100%, 10%-95%, 15%-90%, 20%-85%, 25%-80%, 30%-75%, 35%-70%, 40%-65%, 45%-60%, or 50%-55% compared to a control. Increased expression of a gene may be expression that is increased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

Decreased expression of a gene may be expression that is decreased by 5%-100%, 10%-95%, 15%-90%, 20%-85%, 25%-80%, 30%-75%, 35%-70%, 40%-65%, 45%-60%, or 50%-55% compared to a control. Decreased expression of a gene may be expression that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

An agent may be any agent that modulates expression of a gene in an HSPC. Non-limiting examples of potential agents include small molecules having a molecular weight of less than about 1,000 g/mol; nucleic acid compounds, non-limiting examples of which include a guide RNA (gRNA) used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA), or a combination thereof; a protein (e.g., an antibody); a polypeptide (e.g., including a protein active site); and a nucleic acid aptamer.

An agent may also be a combination of agents. In some embodiments, multiple agents are from the same category (e.g., small molecule, gRNA). In some embodiments, multiple agents are from different categories (e.g., small molecule and gRNA). In some embodiments, a combination of agents includes 1-10, 2-9, 3-8, 4-7, or 5-6 agents. In some embodiments, a combination of agents includes 1, 2, 3, 4, 5, 6, 7, 8, 9, or 10 agents.

In some embodiments, an agent is contained in a library of agents. A library is a collection of agents. A library may contain 10-500,000; 100-100,000; 1,000-50,000; 5,000-25,000 agents. In some embodiments a library contains 10; 25; 50; 75; 100; 200; 300; 400; 500; 600; 700; 800; 900; 1,000; 2,500; 5,000; 7,500; 10,000; 12,500; 15,000; 17,500; 20,000; 22,500; 25,000; 27,500; 30,000; 32,500; 35,000; 37,500; 40,000; 42,500; 45,000; 47,500; 50,000; 52,500; 55,000; 57,500; 60,000; 62,500; 65,000; 67,500; 70,000; 72,500; 75,000; 77,500; 80,000; 82,500; 85,000; 87,500; 90,000; 92,500; 95,000; 97,500; 100,000; or more, agents.

In some embodiments, an agent is a guide RNA (gRNA) used in a CRISPR/Cas genome editing system. CRISPR/Cas genome editing is well-known in the art. (see, e.g., Wang et al., Ann. Rev. Biochem., 2016, 85: 227-264; Pickar-Oliver and Gersbach, Nature Reviews Molecular Cellular Biology, 2019, 20: 490-507; Aldi, Nature Communications, 2018, 9: 1911). In some embodiments, an agent that is a gRNA knocks out (removes) a gene from the genome, decreases expression of the gene from the genome, decreases protein activity, or a combination thereof. An agent that is a gRNA may be 1-10, 2-9, 3-8, 4-7, or 5-6 gRNAs. In some embodiments, an agent that is a gRNA may be 1, 2, 3, 4, 5, 6, 7, 8, 9, or 10 or more gRNAs.

In embodiments, where an agent is a nucleic acid (e.g., gRNA, siRNA, aiRNA), an HSPC expresses a programmable nuclease protein. A programmable nuclease protein may be expressed in an HSPC: following a genomic modification of an HSPC (e.g., to insert a sequence encoding a programmable nuclease protein), following transfection of a sequence encoding a programmable nuclease protein into an HSPC, or by introduction of a polypeptide encoding a programmable nuclease protein into an HSPC.

A programmable nuclease protein is an enzyme that binds to a nucleic acid and is recruited to and cuts a target nucleic acid sequence (e.g., gene). Programmable nuclease proteins may be catalytically active (e.g., cuts a target nucleic acid sequence) or catalytically dead (e.g., binds but does not cut a target nucleic acid sequence).

A programmable nuclease protein may be any programmable nuclease protein known in the art. Non-limiting examples of programmable nuclease proteins include: CRISPR/Cas RNA-guided engineered nucleases; zinc-finger nucleases (ZFNs), and transcription activator-like effector nucleases (TALENs). In some embodiments, a programmable nuclease protein is a CRISPR/Cas nuclease.

CRISPR/Cas nucleases may be any CRISPR/Cas nuclease known in the art. Non-limiting examples of CRISPR/Cas nucleases include: Type I (e.g., Cas3, Cas8a2, Csa5, Cas8b, Cas8c, Cas5, Cas10d, Cse1, Cse2, Csy1, Csy2, Csy3, Cas6f, Csy1/Csy2 fusion, PBPRB1993, PBPRB1992), Type II (e.g., Csn2, Cas12, Csx12), Type III (e.g., Csm2, Cas10, all1473, Cmr5, Cas10, Csx11), or Type IV (e.g., Csf4, RHA1_ro10070). In some embodiments, a programmable nuclease protein is Cas9. Cas9 may be any Cas9 known in the art. Non-limiting examples of Cas9 include: Streptococcus pyogenes Cas9 (spCas9), Staphylococcus aureus Cas9, SpCas9-high fidelity, enhanced nuclease (eSpCas9), hyper-accurate Cas9 (HypaCas9), and FokI-Cas9.

Identifying Genes that Promote Clonal Hematopoiesis

Methods provided herein may be used to identify a gene that promotes clonal hematopoiesis (CH). When a gene promotes CH, its decreased expression decreases the competitive advantage (e.g., proliferation, survival, differentiation) of an HSPC having a genomic modification associated with CH. HSPC proliferation, survival, and/or differentiation may be measured by any method provided herein.

In some embodiments, HSPCs with a genomic modification associated with CH are isolated from a subject. In some embodiments, isolated HSPCs with a genomic modification associated with CH are then contacted with an agent that modulates expression of a gene in the HSPCs. In some embodiments, the isolated HSPCs are contacted with a library of agents. In some embodiments, the contacted HSPCs are then sequenced and baseline expression of a gene is measured. In some embodiments, the contacted HSPCs are then co-cultured with endothelial cells. In some embodiments, a sample of co-cultured HSPCs is then collected and HSPCs are separated from mature hematopoietic cells. In some embodiments, the separated HSPCs and mature hematopoietic cells are measured for expression of the gene. A gene is identified as promoting CH when expression of the gene in HSPCs is decreased compared to a control.

In some embodiments, a genomic modification associated with CH is a substitution, insertion, or deletion in a gene encoding a protein, and the genomic modification results in expression of a variant of the protein that is associated with CH. In some embodiments, a genomic modification is a substitution, insertion, or deletion in a gene encoding an IDH2 protein, a TET2 protein, an ASXL1 protein, and/or a DNMT3A protein.

A gene that promotes CH is a gene that promotes the maintenance of CH, promotes the progression of CH (e.g., into cancer, into cardiovascular disease), or some combination thereof. Promoting the maintenance of CH may be increased proliferation of HSPCs having genomic modifications associated with CH, increased survival of HSPCs having genomic modifications associated with CH, or some combination thereof. Increased proliferation and/or increased survival of HSPCs having genomic modifications associated with CH may be compared to a wild-type HSPC.

An agent may be any agent provided herein. In some embodiments, an agent is contained in a library of agents, which can be a library of any size provided herein.

In some embodiments, HSPCs having a genomic modification associated with CH are contacted with a library of agents that modulate expression of different genes in the HSPCs. In some embodiments, the HSPCs express a programmable nuclease protein (e.g., Cas9), and the library of agents comprises single guide RNAs (sgRNAs). Accordingly, in some embodiments, genomic screening may be used to perform methods of identifying a gene that promotes CH. For example, in some embodiments, the methods involve the use of genome-wide library screening techniques known in the art (see, e.g., Wei, et al. Genome-wide CRISPR/Cas9 library screening identified PHGDH as a critical driver for Sorafenib resistance in HCC. Nat Commun 10, 4681 (2019)), which are applied to a co-culture system described herein.

In some embodiments, HSPCs having a genomic modification associated with CH are contacted with a library of sgRNAs that modulate expression of different genes in the HSPCs, and the contacted HSPCs are cultured with endothelial cells (e.g., BMECs). In some embodiments, the cultured HSPCs are analyzed to determine gene expression levels. For example, in some embodiments, an expression level is determined by sequencing the cultured HSPCs, and determining a copy number for an sgRNA corresponding to a gene in the HSPCs based on the sequencing, where the copy number is indicative of the expression level of the gene. In some embodiments, a decrease in the copy number relative to a control copy number indicates that the gene promotes CH.

In some embodiments, a control copy number is determined by sequencing an aliquot of the HSPCs at an earlier time point in the culturing. In some embodiments, the control copy number is determined by sequencing wild-type HSPCs. For example, in some embodiments, the control copy number is determined by: contacting wild-type HSPCs with the library of sgRNAs, where the wild-type HSPCs do not comprise the genomic modification associated with CH; culturing the contacted wild-type HSPCs with endothelial cells; sequencing the cultured wild-type HSPCs; and determining the control copy number for the sgRNA corresponding to the gene in the wild-type HSPCs based on the sequencing.

Contacting an HSPC having a genomic modification associated with CH with an agent may be by any method known in the art. Non-limiting methods of contacting an HSPC with an agent include: introducing an agent into a cell culture medium, injecting an agent into an HSPC, or some combination thereof.

Sequencing a cell (e.g., HSPCs, mature hematopoietic cells) may be by any method known in the art. Non-limiting methods of sequencing a cell include: single cell sequencing, single cell RNA sequencing, and single cell DNA sequencing. Measuring gene expression may be by any method known in the art including: real time quantitative PCR (RT-qPCR), real time PCR (RT-PCR), chromatin immunoprecipitation (ChIP), Northern blot, and Southern blot.

HSPCs that have been contacted with an agent may be cultured with any endothelial cells provided herein. In some embodiments, HSPCs that have been contacted with an agent are cultured with bone marrow endothelial cells (BMECs). HSPCs that have been co-cultured with endothelial cells are collected, and HSPCs are separated from mature hematopoietic cells. Separating HSPCs from mature hematopoietic cells may be by any method known in the art. Non-limiting methods of separating HSPCs from mature hematopoietic stem cells include: FACS (e.g., SCA-1⁺, c-Kit⁺), MACS, Rhodamine-123 staining, and Hoescht 33342 staining.

A gene is identified as promoting CH when expression of the gene in HSPCs is decreased after being contacted with an agent compared to a control. A control is an HSPC with a genomic modification associated with CH that is not contacted with an agent, a wild-type HSPC contacted with the agent, or an HSPC with a genomic modification associated with CH that is contacted with an agent that is not known to modulate gene expression. Decreased gene expression may be gene expression that is decreased by 5%-100%, 10%-95%, 15%-90%, 20%-85%, 25%-80%, 30%-75%, 35%-70%, 40%-65%, 45%-60%, or 50%-55% or more compared to a control. Decreased gene expression may be gene expression that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

In some embodiments, the following steps or the method are performed multiple times: 1) a sample of co-cultured HSPCs is then collected and HSPCs are separated from mature hematopoietic cells; 2) the separated HSPCs and mature hematopoietic cells are measured for expression of the gene; and 3) a gene is identified as promoting CH when expression of the gene in HSPCs is decreased compared to a control. These method steps may be repeated 1-20 times, 2-19 times, 3-18 times, 4-17 times, 5-16 times, 6-15 times, 7-14 times, 8-13 times, 9-12 times, or 10-11 times. In some embodiments, these method steps are repeated 1, 2, 3, 4, 5, 6, 7, 8, 9, 10, 11, 12, 13, 14, 15, 16, 17, 18, 19, 20, or more, times.

A cultured HSPC that has been contacted with agent may be transplanted into a subject. This transplantation into a subject will allow continued study of CH in vivo following ex vivo study. Thus, in some embodiments, the present disclosure provides a combination ex vivo and in vivo platform for studying CH.

After transplantation of cultured HSPCs that have been contacted with an agent into a subject, a sample of the transplanted HSPCs is collected and HSPCs are separated from mature hematopoietic cells in the sample. Transplanted HSPCs and mature hematopoietic cells are then sequenced and gene expression is measured. A gene is identified as promoting CH when expression of the gene in the transplanted HSPCs is decreased compared to a control.

Transplanting cultured HSPCs (e.g., HSPCs and any mature hematopoietic cells differentiated therefrom) having a genomic modification associated with CH may be by any method known in the art, including, but not limited to: bone marrow thymus (BMT) engraftment and umbilical vein engraftment. In some embodiments, transplanted HSPCs are autologous. In some embodiments, transplanted HSPCs are allogenic.

A subject may be any subject provided herein. In some embodiments, a subject is a mouse.

In some embodiments, the following steps or the method are performed multiple times: 1) a sample of transplanted HSPCs is then collected and HSPCs are separated from mature hematopoietic cells; 2) the separated HSPCs and mature hematopoietic cells are measured for expression of the gene; and 3) a gene is identified as promoting CH when expression of the gene in HSPCs is decreased compared to a control. These method steps may be repeated 1-20 times, 2-19 times, 3-18 times, 4-17 times, 5-16 times, 6-15 times, 7-14 times, 8-13 times, 9-12 times, or 10-11 times. In some embodiments, these method steps are repeated 1, 2, 3, 4, 5, 6, 7, 8, 9, 10, 11, 12, 13, 14, 15, 16, 17, 18, 19, 20, or more, times.

### Identifying an Inhibitor of Clonal Hematopoiesis (CH)

Provided herein, in some aspects, is a method of identifying an inhibitor of CH comprising: contacting HSPCs having a genomic modification associated with CH with a test compound; sequencing the contacted HSPCs and measuring baseline expression of a gene that is required for CH; culturing the contacted HSPCs with endothelial cells; collecting a sample of cultured, contacted cells and separating HSPCs from mature hematopoietic cells; sequencing the separated HSPCs and mature hematopoietic cells and measuring survival and proliferation of HSPCs and mature hematopoietic cells; and identifying the test compound as an inhibitor of CH when the survival or proliferation of HSPCs contacted with the test compound is decreased compared to a control.

It will be understood that an HSPC having a genomic mutation associated with CH also includes any mature hematopoietic stem cells that differentiate from the HSPC.

In some embodiments, a genomic modification associated with CH is a substitution, insertion, or deletion in a gene encoding a protein, and the genomic modification results in expression of a variant of the protein that is associated with CH. In some embodiments, a genomic modification is a substitution, insertion, or deletion in a gene encoding an IDH2 protein, a TET2 protein, an ASXL1 protein, and/or a DNMT3A protein.

A test compound may be any test compound that modulates survival, proliferation, and/or differentiation of HSPCs. Non-limiting examples of potential test compounds include small molecules having a molecular weight of less than about 1,000 g/mol; nucleic acid compounds, non-limiting examples of which include a guide RNA (gRNA) used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA), or a combination thereof; a protein (e.g., an antibody); a polypeptide (e.g., including a protein active site); and a nucleic acid aptamer.

A test compound may also be a combination of test compounds. In some embodiments, multiple test compounds are from the same category (e.g., small molecule, gRNA). In some embodiments, multiple test compounds are from different categories (e.g., small molecule and gRNA). In some embodiments, a combination of test compounds includes 1-10, 2-9, 3-8, 4-7, or 5-6 test compounds. In some embodiments, a combination of test compounds includes 1, 2, 3, 4, 5, 6, 7, 8, 9, or 10 test compounds.

In some embodiments, a test compound is contained in a library of test compounds. A library is a collection of test compounds. A library may contain 10-500,000; 100-100,000; 1,000-50,000; 5,000-25,000 test compounds. In some embodiments a library contains 10; 25; 50; 75; 100; 200; 300; 400; 500; 600; 700; 800; 900; 1,000; 2,500; 5,000; 7,500; 10,000; 12,500; 15,000; 17,500; 20,000; 22,500; 25,000; 27,500; 30,000; 32,500; 35,000; 37,500; 40,000; 42,500; 45,000; 47,500; 50,000; 52,500; 55,000; 57,500; 60,000; 62,500; 65,000; 67,500; 70,000; 72,500; 75,000; 77,500; 80,000; 82,500; 85,000; 87,500; 90,000; 92,500; 95,000; 97,500; 100,000; or more, test compounds.

A test compound is identified as an inhibitor of CH if it decreases survival, proliferation, and/or differentiation of an HSPC having a genomic modification associated with CH compared with a control.

HSPC proliferation may be measured by any method provided herein. HSPC survival may be measured by any method provided herein. HSPC differentiation may be measured by any method provided herein. A control may be an HSPC having a genomic modification associated with CH that is not contacted with the test compound or an HSPC that does not have a genomic modification associated with CH (e.g., a wild-type HSPC). Decreased HSPC survival is survival that is decreased by 5%-100%, 10%-95%, 15%-90%, 20%-85%, 25%-80%, 30%-75%, 35%-70%, 40%-65%, 45%-60%, or 50%-55% compared to a control. Decreased HSPC survival may be survival that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

Decreased HSPC proliferation is proliferation that is decreased by 5%-100%, 10%-95%, 15%-90%, 20%-85%, 25%-80%, 30%-75%, 35%-70%, 40%-65%, 45%-60%, or 50%-55% compared to a control. Decreased HSPC proliferation may be proliferation that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

Decreased HSPC differentiation into a mature hematopoietic cell is differentiation that is decreased by 5%-100%, 10%-95%, 15%-90%, 20%-85%, 25%-80%, 30%-75%, 35%-70%, 40%-65%, 45%-60%, or 50%-55% compared to a control. Decreased HSPC differentiation may be proliferation that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

### Inhibiting Clonal Hematopoiesis

Provided herein, in some aspects, is a method of inhibiting clonal hematopoiesis (CH) comprising: contacting an HSPC, the HSPC comprising a genomic modification associated with CH, with an agent that decreases activity of a product of a gene encoding a histone 3, lysine 9 (H3K9) demethylase.

In some embodiments, a genomic modification associated with CH is a substitution, insertion, or deletion in a gene encoding a protein, and the genomic modification results in expression of a variant of the protein that is associated with CH. In some embodiments, a genomic modification is a substitution, insertion, or deletion in a gene encoding an IDH2 protein, a TET2 protein, an ASXL1 protein, and/or a DNMT3A protein.

Histone modifications are key epigenetic regulatory features with important roles in many cellular events. Lysine methylations mark various sites on the tail and globular domains of histones, and their levels are precisely balanced by the action of methyltransferases and demethylases. Misregulation of histone lysine methylation is implicated in numerous diseases or disorders, including cancers, aging, and developmental defects.

H3K9 methylation is a well-known indicator of silenced transcription and heterochromatin structure. Thus, proteins that methylate H3K9 will inhibit gene expression and proteins that demethylate H3K9 will promote gene expression. An agent that decreases activity of a product encoded by a gene encoding a H3K9 demethylase will therefore inhibit gene expression.

Decreased activity of a product encoded by a gene encoding a H3K9 demethylase may be activity that is decreased by 5%-100%, 10%-95%, 15%-90%, 20%-85%, 25%-80%, 30%-75%, 35%-70%, 40%-65%, 45%-60%, or 50%-55% compared to a control. In some embodiments, decreased activity of a product encoded by a gene encoding a H3K9 demethylase is activity that is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

In some embodiments, an H3K9 demethylase whose activity is decreased is a JHDM2 family protein, a JHDM3 family protein, Rph1, CG31123, CG15835, CG33182, or a PHF8 family protein. In some embodiments, a H3K9 demethylator is a JHDM2 family protein. JHDM2 family proteins JHDM2A (KDM3A, JMJD1, JMJD1A), JHDM2B (JMJD1B, KDM3B), and JHDM2C (KDM3C, TRIP8) demethylate mono-methylated H3K9 (H3K9me1) and di-methylated H3K9 (H3K9me2) and regulate transcription activation. In some embodiments, a H3K9 demethylator is a JHDM3 family protein. JHDM3 family proteins JHDM3A (KDM4A, JMJD2A, JMJD2), JHDM3B (KDM4B, JMJD2B), JHDM3C (KDM4C, JMJD2C), JHDM3D (KDM4D, JMJD2D), and JHDM3E (KDM4E, JMJD2E) demethylate H3K9me2 and tri-methylated H3K9 (H3K9me3).

In some embodiments, methods provided herein include contacting a HSPC with an agent that decreases the activity of a product encoded by a gene encoding a lysine demethylase 3B (KDM3B) protein. KDM3B (JMJD1B) is a Jumonji C domain-containing protein that catalyzes demethylation of histone 3, lysine 9, methyl 1 (H3K9me1) and H3K9me2, resulting in activation of gene transcription. Moreover, KDM3B demethylates H3K9me2, which alters chromatin modification and dynamically regulates the gene expression of differentiated cells and the proliferation of cells.

KDM3B is conserved in human (Gene ID: 51780), mouse (Gene ID: 277250), rat (Gene ID: 682469), zebrafish (Gene ID: 326643), non-human primate (Gene IDs: 462095, 716648), chicken (Gene ID: 100859412), dog (Gene ID: 474695), cat (Gene ID: 101087899), and pig (Gene ID: 100515884).

In some embodiments, KDM3B is human KDM3B encoding a human KDM3B protein. There are 3 isoforms of human KDM3B protein due to alternative splicing of the mRNA. In some embodiments, a KDM3B protein is human KDM3B isoform 1 (Uniprot ID: Q7LBC6-1), which contains amino acids 1-1,761. In some embodiments, a KDM3B protein is human KDM3B isoform 2 (Uniprot ID: Q7LBC6-2), which is missing amino acids 1-344 with respect to human KDM3B isoform 1. In some embodiments, a KDM3B protein is human KDM3B isoform 3 (Uniprot ID: Q7LBC6-3), which is missing amino acids 1-1,002 with respect to human KDM3B isoform 1.

An agent that decreases the activity of a product encoded by a gene encoding a histone 3, lysine 9 (H3K9) demethylase may be any agent known in the art. Non-limiting examples of potential agents that decrease activity of a product encoded by a gene encoding a H3K9 demethylase include small molecules having a molecular weight of less than about 1,000 g/mol; nucleic acid compounds, including, for example, a guide RNA (gRNA) used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA); a protein (e.g., an antibody); a polypeptide (e.g., containing the H3K9 demethylase active site); a nucleic acid aptamer; or a combination thereof.

As used herein, an agent that decreases the activity of a product encoded by a gene can refer to any agent that directly targets the gene or the product (e.g., by targeting at the level of the genome, transcriptome, or proteome). For example, in some embodiments, an agent is a genome-editing agent (e.g., sgRNA) that targets a gene to decrease activity of a product encoded by the gene. In some embodiments, the product encoded by the gene is a protein, and an agent is an antisense oligonucleotide that targets an mRNA encoding the protein. In some embodiments, an agent is an inhibitor of the protein.

Accordingly, in some embodiments, an agent described herein is a protein inhibitor that decreases the activity of a protein encoded by a gene encoding a H3K9 demethylase. In some embodiments, a protein inhibitor is an enzymatic inhibitor that binds to a protein (e.g., H3K9 demethylase) to decrease enzymatic activity of the protein (e.g., through competitive or non-competitive means of enzymatic inhibition). In some embodiments, a protein inhibitor is a mediator of protein degradation that mediates targeted degradation of a protein (e.g., H3K9 demethylase). In some embodiments, the mediator of protein degradation is a proteolysis-targeted chimera (PROTAC) molecule. Techniques for the design and use of molecules for targeted protein degradation are known in the art, see, e.g., Békés, et al. PROTAC targeted protein degraders: the past is prologue. Nat Rev Drug Discov 21, 181-200 (2022); and Zeng, et al. Exploring Targeted Degradation Strategy for Oncogenic KRAS^{G12C} Cell Chem Biol 27, 19-31 (2020).

In some embodiments, a KDM3B inhibitor is PFI-63, PFI-90, JIB-04, IOX1, DMOG, SD-70, ML324, KDM5-C70, PBIT, KDOHP64a, KDOQZ5, IOX2, KDMA83, KDMOBP69, NSC636819, a single guide RNA (sgRNA), a small activating RNA (saRNA) as described in JP 2018512876, which is incorporated by reference herein.

In some embodiments, methods provided herein include contacting a HSPC with an agent that decreases the activity of a product encoded by a gene encoding a Jumonji Domain Containing 1C (JMJD1C) protein. JMJD1C (KDM3C) is a Jumonji C domain-containing protein that catalyzes demethylation of histone 3, lysine 9, methyl 1 (H3K9me1) and H3K9me2, resulting in activation of gene transcription.

JMJD1C is conserved in human (Gene ID: 221037), mouse (Gene ID: 108829), rat (Gene ID: 171120), non-human primate (Gene IDs: 697630, 450489), chicken (Gene ID: 423655), dog (Gene ID: 479221), and pig (Gene ID: 100157328).

In some embodiments, JMJD1C is human JMJD1C encoding a human JMJD1C protein. There are 3 isoforms of human JMJD1C protein due to alternative splicing of the mRNA. In some embodiments, a JMJD1C protein is human JMJD1C isoform 1 (Uniprot ID: Q15652-1), which contains amino acids 1-2,540. In some embodiments, a JMJD1C protein is human JMJD1C isoform 2 (Uniprot ID: Q15652-2), which is missing amino acids 1-219 and 1700-2540 with respect to human JMJD1C isoform 1. In some embodiments, a JMJD1C protein is human JMJD1C isoform 3 (Uniprot ID: Q15652-3), which is missing amino acids 1-182 with respect to human JMJD1C isoform 1.

In some embodiments, a JMJD1C inhibitor is GSK-J4 HCl, JDI-4, JDI-12, JDI-16, JIB-04, IOX1, DMOG, SD-70, ML324, KDM5-C70, PBIT, KDOHP64a, KDOQZ5, IOX2, KDMA83, KDMOBP69, NSC636819, a sgRNA, a small activating RNA (saRNA) as described in JP 2018512876, which is incorporated by reference herein.

Inhibiting CH may be measured by any method provided herein. In some embodiments, inhibiting CH is decreased survival, proliferation, expression of a gene that is required for CH, or some combination thereof compared with a control. A control may be a HSPC having a genomic modification associated with CH that is not contacted with an agent that decreases the activity of a gene product encoded by a gene encoding a H3K9 demethylase or a wild-type HSPC that does not have a genomic modification associated with CH.

Inhibiting CH may be decreased survival, proliferation, expression of a gene required for CH, or some combination thereof that is decreased by 5%-100%, 10%-95%, 15%-90%, 20%-85%, 25%-80%, 30%-75%, 35%-70%, 40%-65%, 45%-60%, or 50%-55% compared to a control. In some embodiments, inhibiting CH is decreased survival, proliferation, expression of a gene required for CH, or some combination thereof is decreased by 5%, 10%, 15%, 20%, 25%, 30%, 35%, 40%, 45%, 50%, 55%, 60%, 65%, 70%, 75%, 80%, 85%, 90%, 95%, or 100%, or more, compared to a control.

### EXAMPLE

### Example 1: Identification and Verification of Potential Therapeutic Targets in Mutant Clonal Hematopoiesis (CH)

Unbiased large-scale chemical and genetic screens to identify targets in CH-mutant cells has not been possible to date due to: 1) a lack of scale or throughput with current in vivo CH-mutant systems, 2) the inability of transformed cell lines to accurately recapitulate CH-mutant disease, and 3) a lack of cell culture conditions suitable for maintaining primary, genetically defined murine hematopoietic stem and progenitor cells (HSPCs) ex vivo.

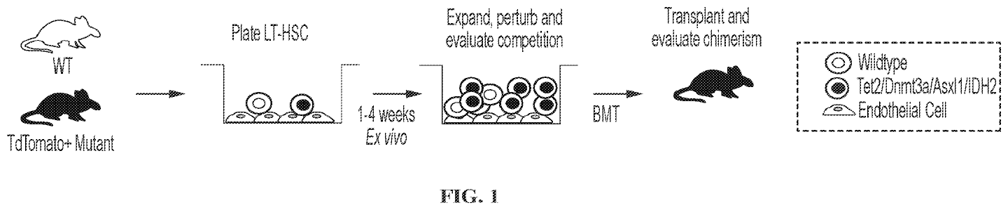

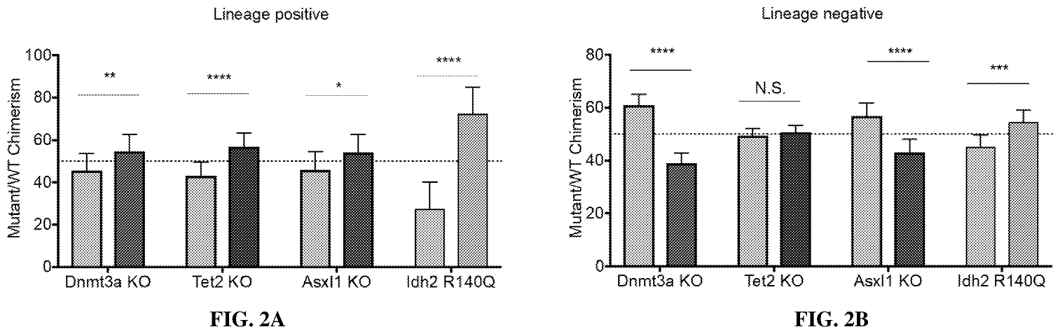

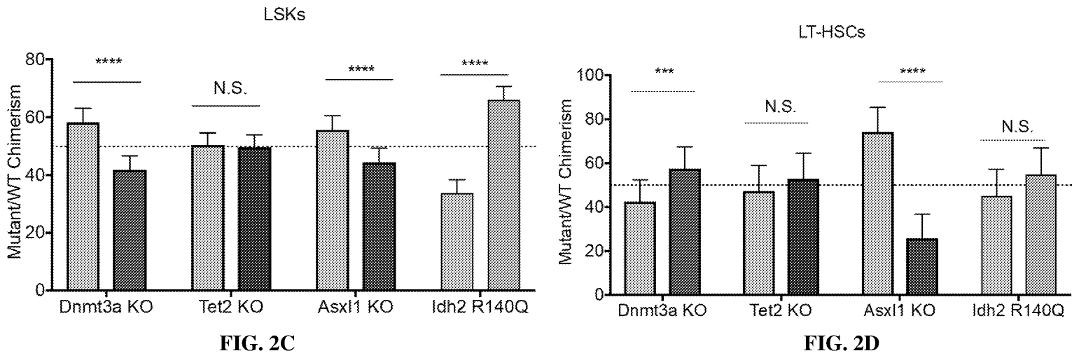

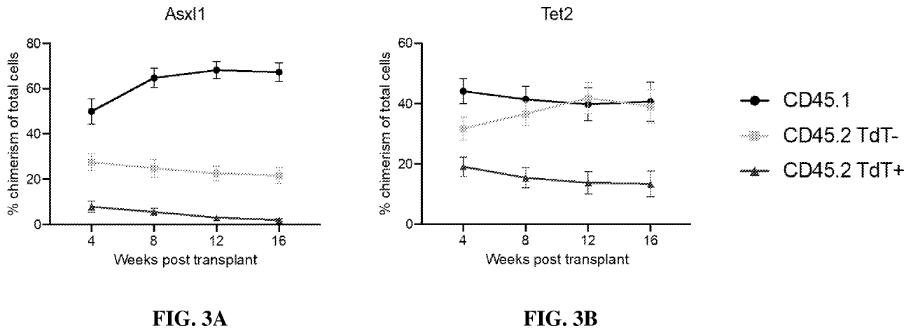

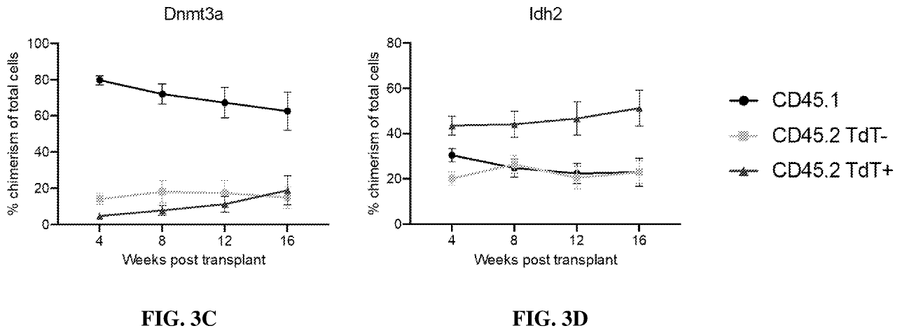

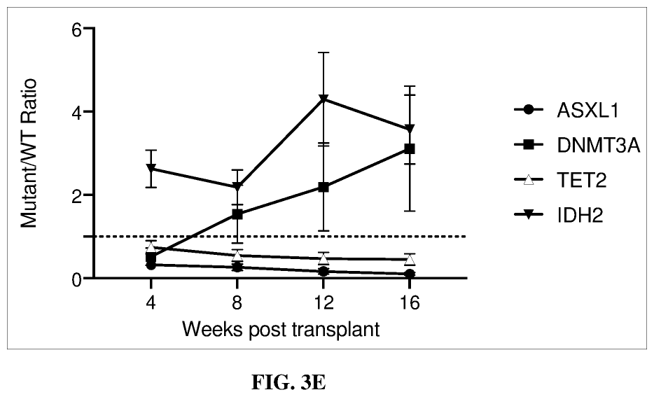

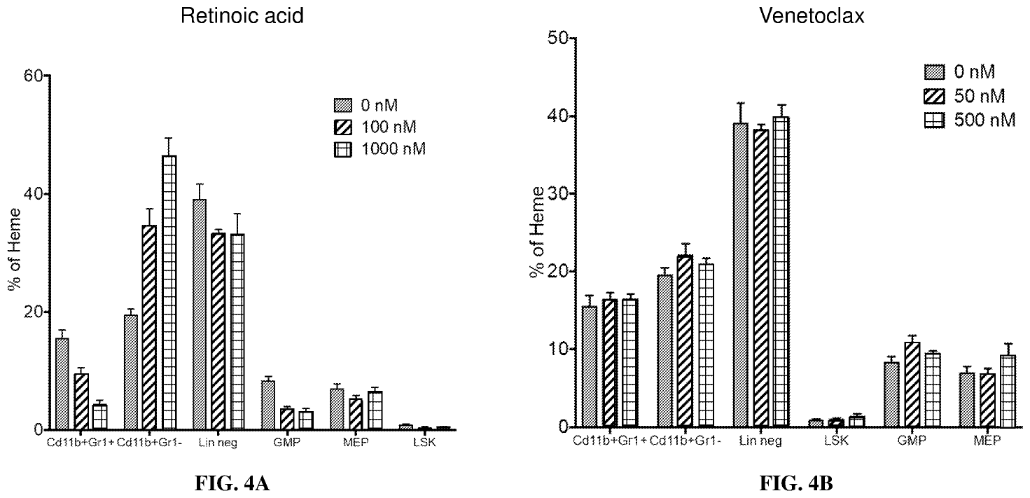

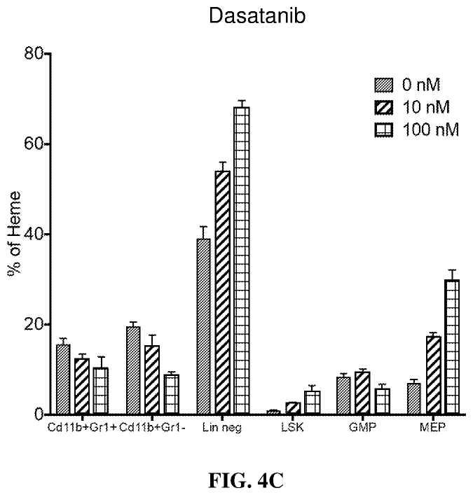

A platform was adopted whereby primary murine HSPCs are co-cultured with bone marrow endothelial cells that allow for maintenance and expansion of HSPCs (FIG. 1). CH-mutant HSPCs co-cultured in this platform demonstrated phenotypes ex vivo over a period of 7-14 days that faithfully recapitulate in vivo phenotypes that ordinarily take months to manifest (FIGS. 2A-2D). HSPCs co-cultured with BMECs in this platform can be transplanted into mice following culture and demonstrate phenotypes consistent with previous murine and human cell findings upon transplantation (FIGS. 3D-3E). Additionally, HSPCs co-cultured with BMECs demonstrate cell-type specific effects when treated with pro-differentiation agents, agents that do not affect differentiation, and anti-differentiation agents (FIGS. 4A-4C). Thus, the adopted HSPC-BMEC co-culture platform is a robust system to study genetically defined murine HSPCs with CH genomic modifications.

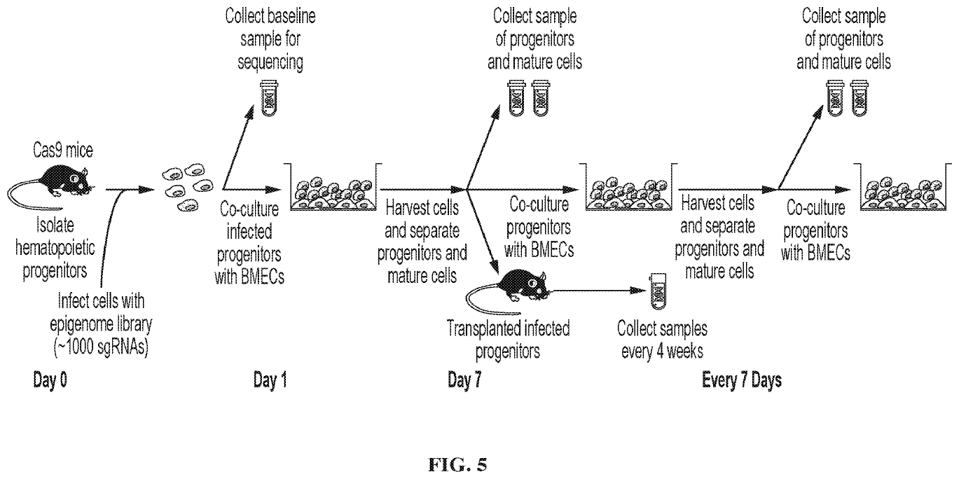

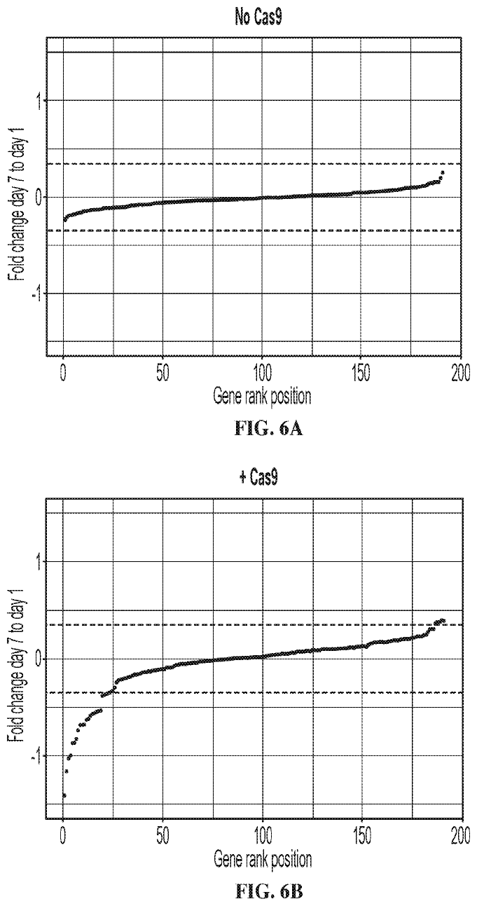

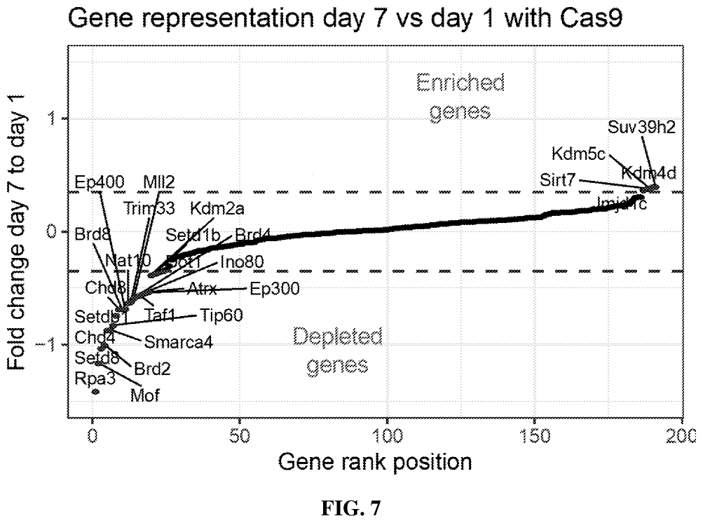

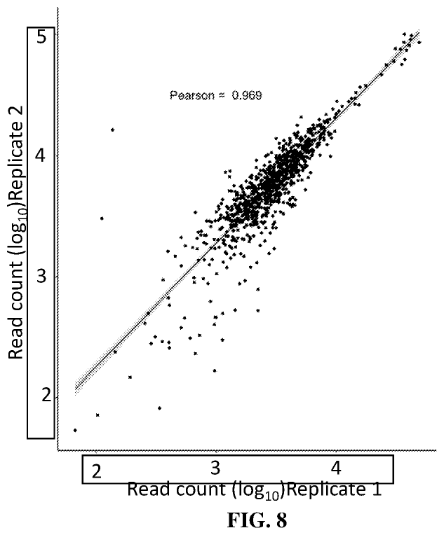

The HSPC-BMEC co-culture platform was used to characterize hematopoiesis gene-dependencies using an unbiased CRISPR/Cas9 genomic screen (FIG. 5). The use of a CRISPR/Cas9 screen in primary genetically defined murine HSPCs has never been reported, likely due to the technical challenges associated with culturing and expanding HSPCs. Wild-type (WT) HSPCs show a range of gene-dependencies based upon the presence of Cas9 (FIGS. 6A-6B), including those gene-dependencies known in native hematopoiesis or across multiple cellular contexts (FIG. 7). These results were reproducible across multiple experimental replicates (FIG. 8).

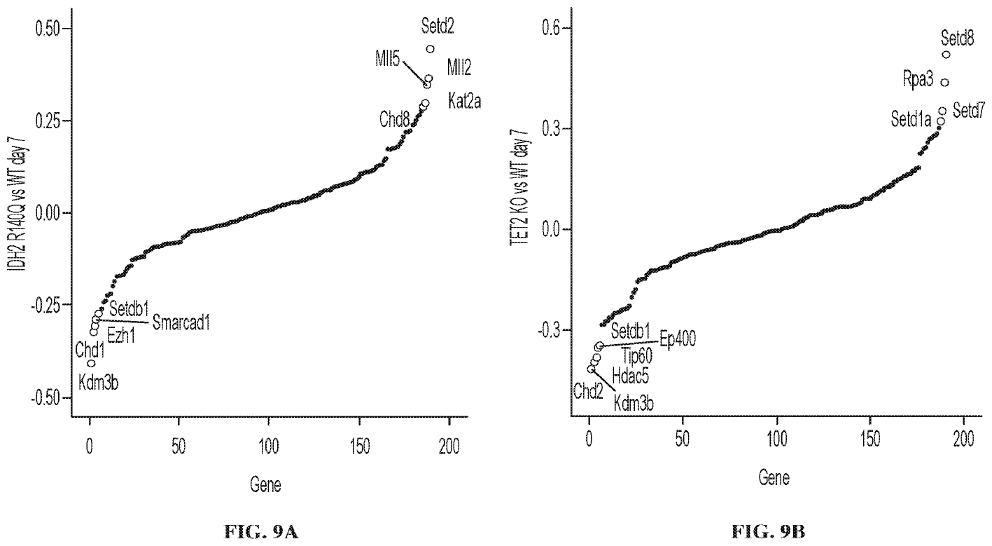

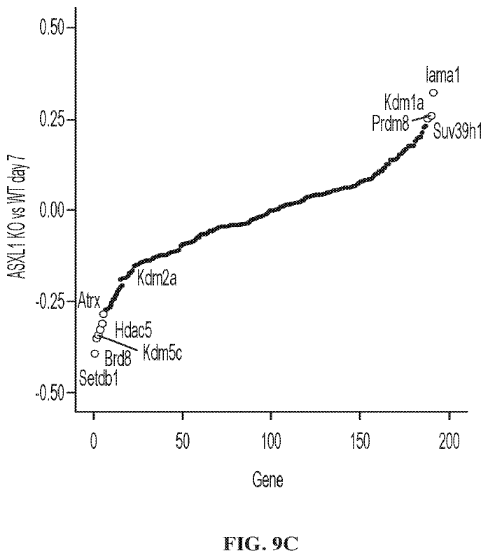

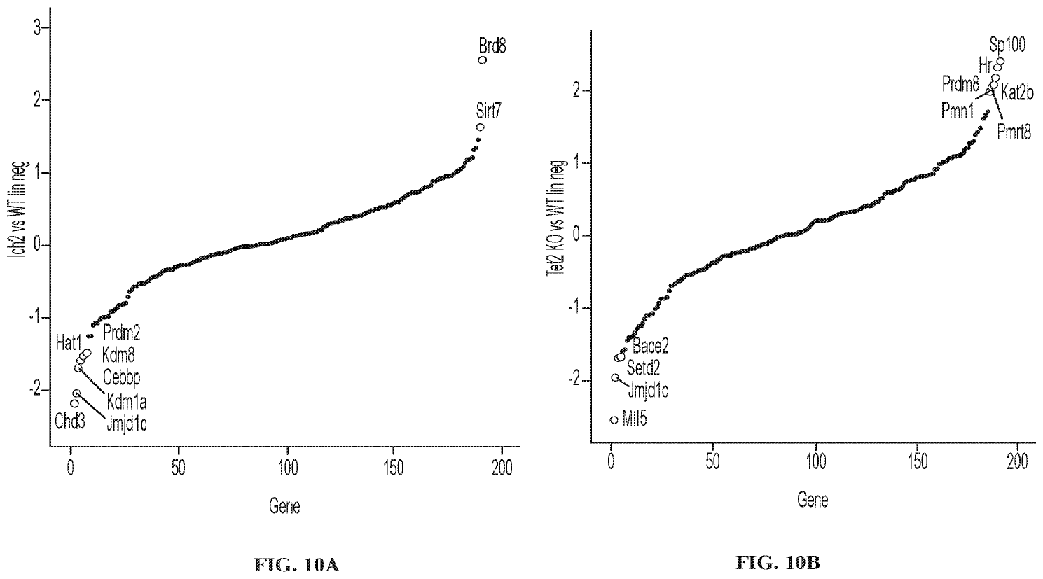

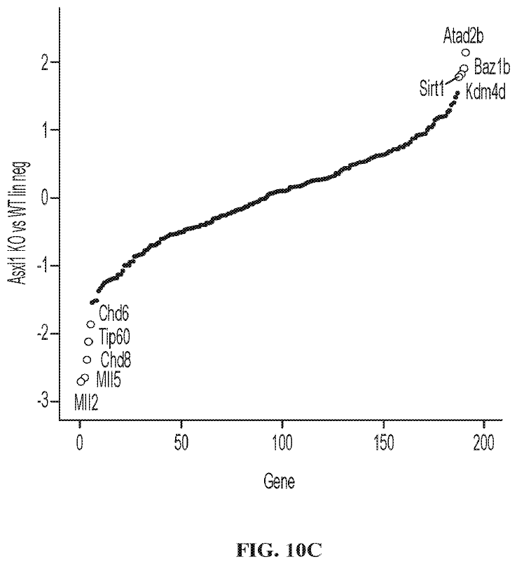

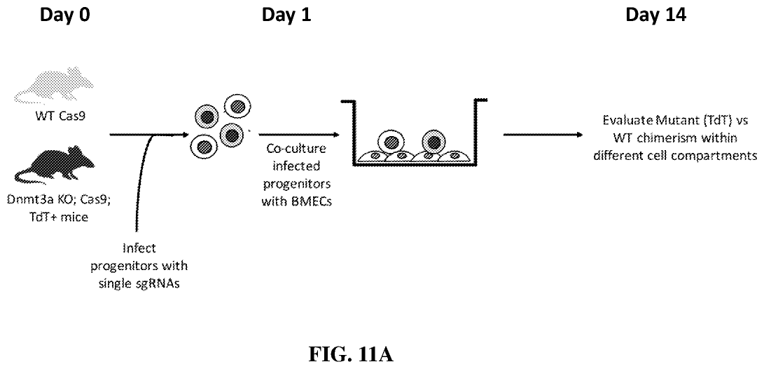

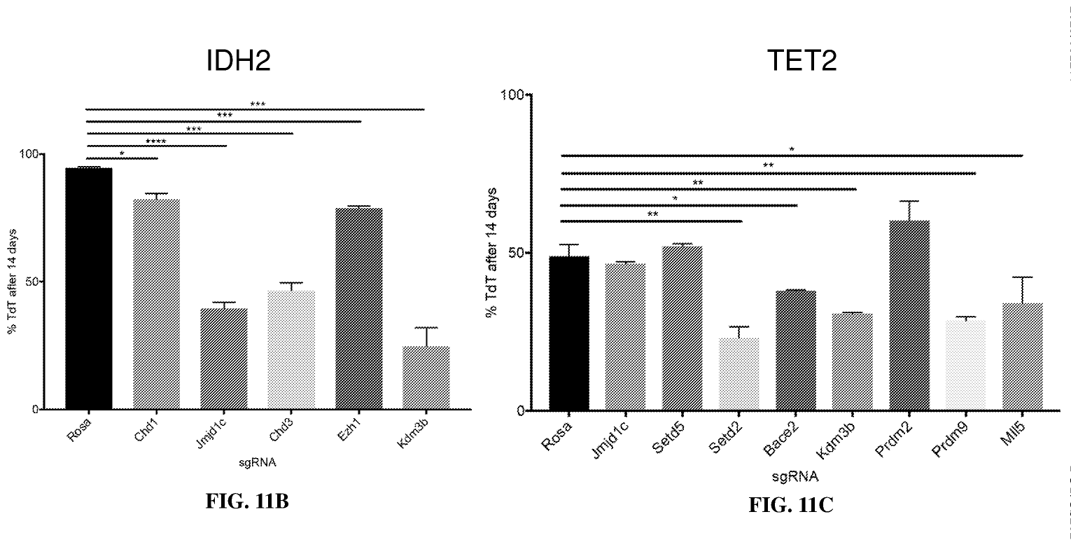

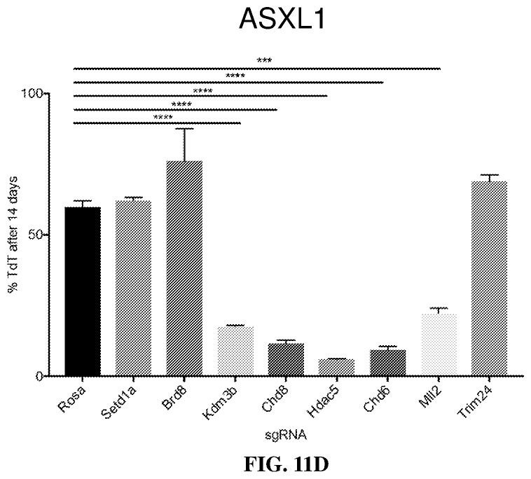

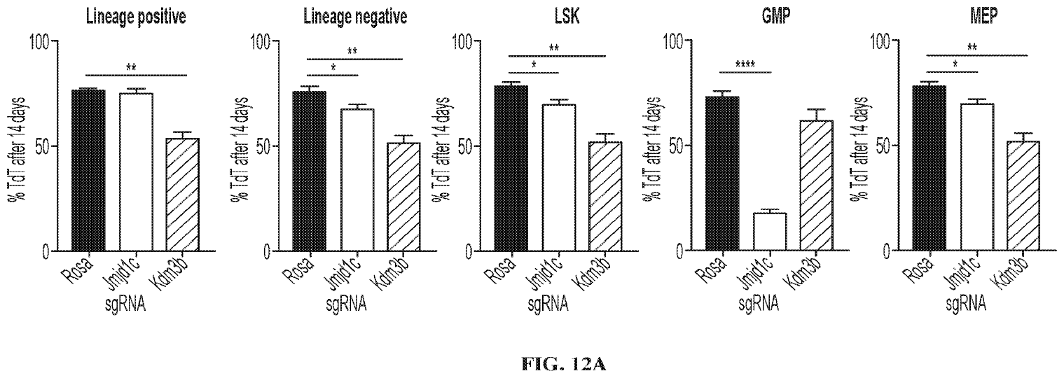

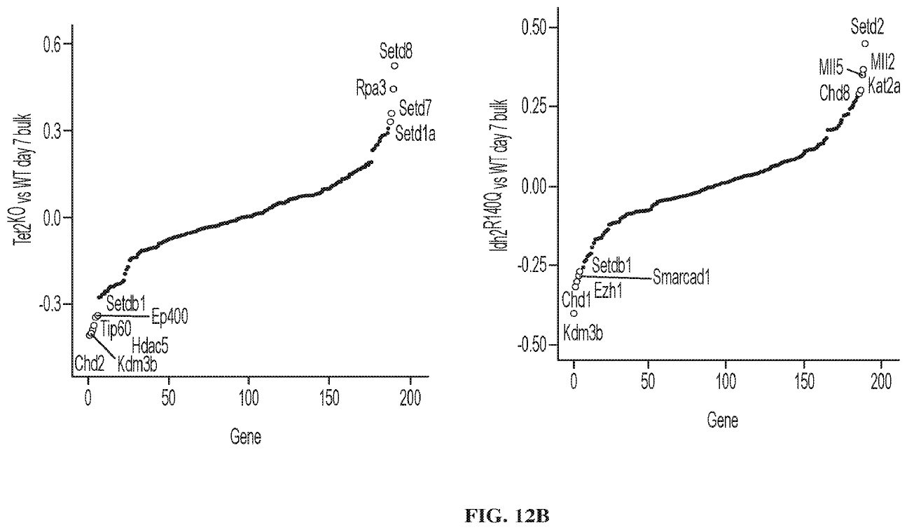

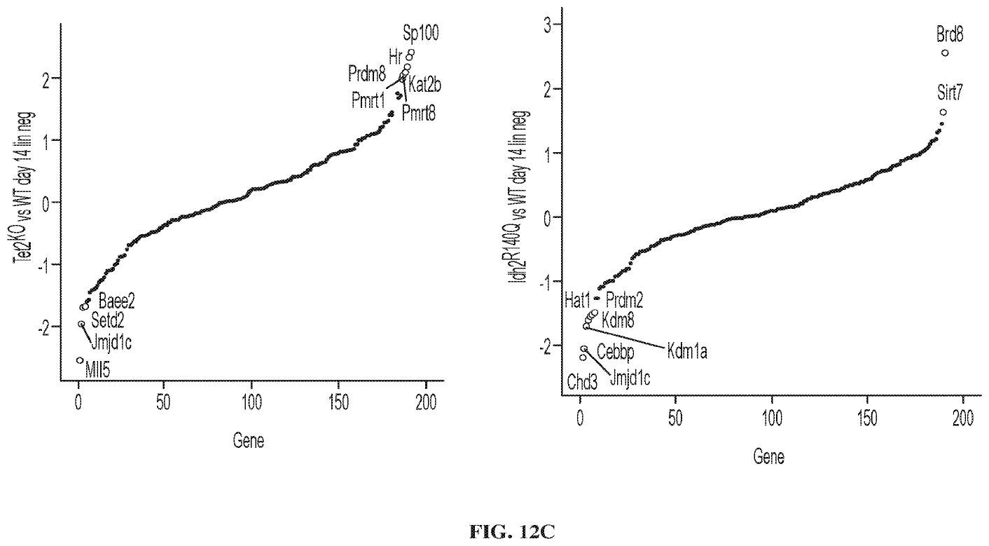

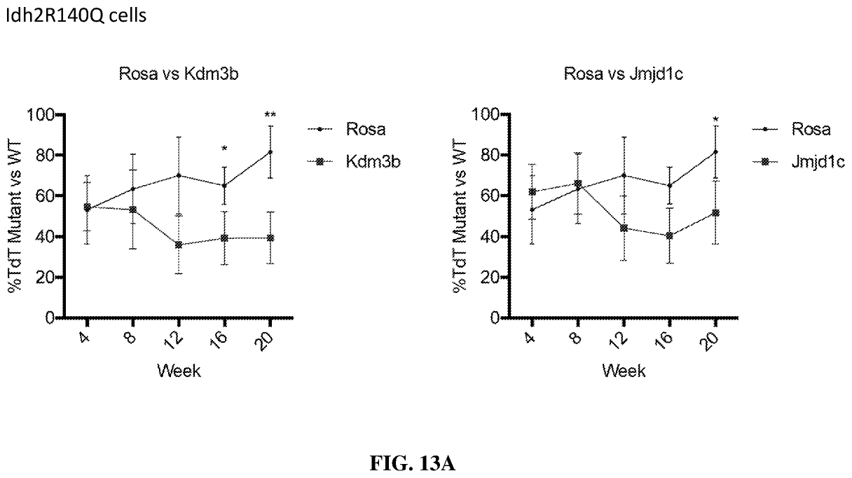

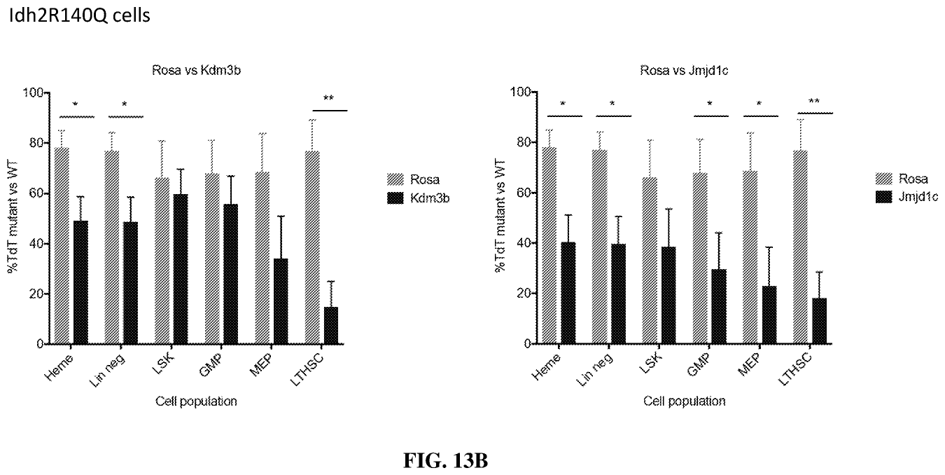

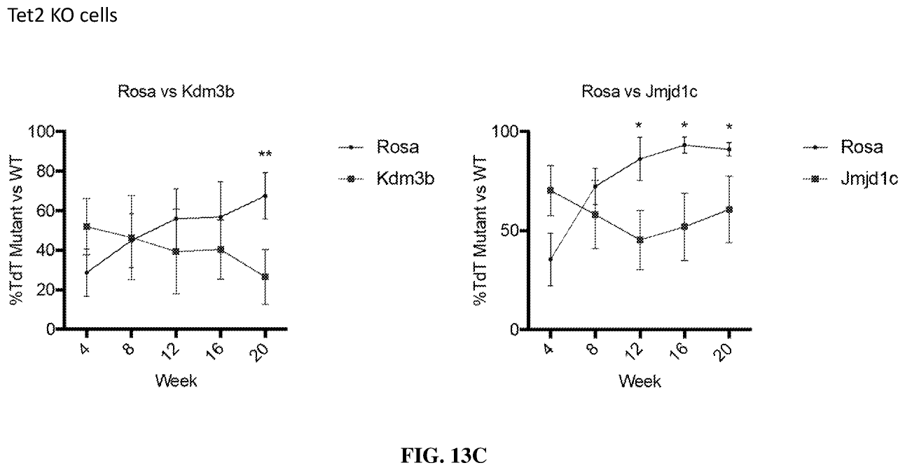

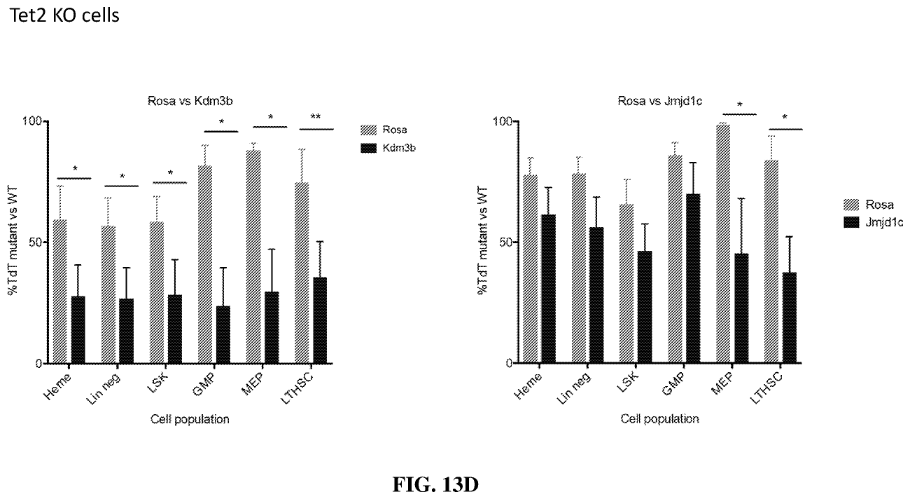

CRISPR/Cas9 screens were performed in the commonly-mutated CH genes Tet2 (Tet2 knock-out, Tet2 KO), Asxl1 (Asxl1 knock-out, Asxl1 KO), and IDH2 (IDH2 R140Q) HSPCs. A range of CH mutant-specific gene dependencies were identified in total (bulk) cell populations and in HSPCs (lineage negative, lin neg) (FIGS. 9A-9C, 10A-10C). A number of these CH-mutant specific gene dependencies were validated in single guide RNA (sgRNA) studies and it was confirmed that these genes are selectively essential for CH-mutant specific HSPCs over WT HSPCs (FIGS. 11A-11D). For IDH2 mutant cells, validated gene dependencies are Chd1, Jmjd1c, Chd3, Ezh1, and Kdm3b. For Tet2 mutant cells, validated gene dependencies are Jmjd1c, Setd2, Bace2, Kdmb, Pdm9, and M115. For Asxl1 mutant cells, validated gene dependencies are Kdm3b, Chd8, Hdac5, Chd6, and M112 (FIGS. 11B-11D). Of particular interest, the Jumanji-domain containing family proteins Jmjd1b (Kdm3b) and Jmjd1c (Kdmc) are top hits and validated gene dependencies in both Tet2 and IDH2-mutant HSPCs (FIGS. 12A-12C). In vivo murine single sgRNA validations of Kdm3b and Jmjd1c also confirmed genotype-selective dependencies of Kdm3b and Jmjd1c in Tet2- and IDH2-mutant HSPCs in both the blood, bone marrow, and stem cell compartments (FIGS. 13A-13D).

Overall, these results demonstrate that the HSPC-BMEC co-culture platform is a robust system for identifying and validating potential CH-mutant specific gene dependencies. Once identified and validated, the relevant genes and proteins may be targeted to treat or prevent CH and other downstream disorders, including myelodysplastic syndrome and acute myeloid leukemia (AML). Thus, this HSPC-BMEC co-culture platform may be used to not only identify gene targets of CH, but also to screen for and develop inhibitors against gene targets required for CH.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of identifying a gene that promotes clonal hematopoiesis (CH), the method comprising:
a) contacting hematopoietic stem cells (HSPCs) with an agent that modulates expression of a gene in the HSPCs, wherein the HSPCs comprise a genomic modification associated with CH;
b) culturing the contacted HSPCs with endothelial cells; and
c) determining an expression level of the gene in the cultured HSPCs, wherein a decrease in the expression level relative to a control indicates that the gene promotes CH.

2. The method of claim 1, wherein the genomic modification comprises a substitution, insertion, or deletion in a gene encoding a protein, and the genomic modification results in expression of a variant of the protein that is associated with CH.

3. The method of claim 1 or claim 2, wherein the genomic modification is a substitution, insertion, or deletion in a gene encoding an IDH2 protein, a TET2 protein, an ASXL1 protein, and/or a DNMT3A protein.

4. The method of any one of claims 1-3, wherein the agent that modulates expression of the gene is a small molecule, a nucleic acid, a polypeptide, or a protein.

5. The method of any one of claims 1-4, wherein the agent is contained in a library of agents, and the contacting comprises contacting the HSPCs with the library of agents.

6. The method of any one of claims 1-5, further comprising:
d) transplanting the HSPCs into a subject; and
e) determining a second expression level of the gene in the transplanted HSPCs, wherein a decrease in the second expression level relative to a control indicates that the gene promotes CH.

7. The method of any one of claims 1-6, wherein step (c) is performed at least twice during the culturing of the contacted HSPCs.

8. The method of any one of claims 1-7, wherein the endothelial cells are bone marrow endothelial cells (BMECs).

9. The method of any one of claims 1-8, wherein the HSPCs express a programmable nuclease protein.

10. The method of claim 9, wherein the programmable nuclease protein is Cas9.

11. The method of any one of claims 1-10, wherein the control comprises an expression level of the gene in wild-type HSPCs that do not comprise the genomic modification.

12. The method of any one of claims 5-11, wherein the library of agents comprises single guide RNAs (sgRNAs).

13. The method of any one of claims 1-12, wherein the contacting of (a) comprises:
contacting the HSPCs with a library of sgRNAs that modulate expression of different genes in the HSPCs.

14. The method of claim 13, wherein the determining of (c) comprises:
sequencing the cultured HSPCs; and
determining a copy number for an sgRNA corresponding to a gene in the HSPCs based on the sequencing, wherein the copy number is indicative of the expression level of the gene.

15. The method of claim 14, wherein a decrease in the copy number relative to a control copy number indicates that the gene promotes CH.

16. The method of claim 15, wherein the control copy number is determined by a method comprising:
contacting wild-type HSPCs with the library of sgRNAs, wherein the wild-type HSPCs do not comprise the genomic modification associated with CH;
culturing the contacted wild-type HSPCs with endothelial cells;
sequencing the cultured wild-type HSPCs; and
determining the control copy number for the sgRNA corresponding to the gene in the wild-type HSPCs based on the sequencing.

17. The method of claim 15, wherein the control copy number is determined by sequencing an aliquot of the HSPCs at an earlier time point in the culturing.

18. A cell culture comprising hematopoietic stem cells (HSPCs) and endothelial cells, wherein the HSPCs comprise:
a) a first HSPC comprising a genomic modification associated with clonal hematopoiesis (CH); and
b) a second HSPC that does not comprise the genomic modification associated with CH.

19. The cell culture of claim 18, wherein the endothelial cells are bone marrow endothelial cells (BMECs).

20. The cell culture of claim 18 or claim 19, wherein the genomic modification comprises a substitution, insertion, or deletion in a gene encoding a protein, and the genomic modification results in expression of a variant of the protein that is associated with CH.

21. The cell culture of any one of claims 18-20, wherein the genomic modification is a substitution, insertion, or deletion in a gene encoding an IDH2 protein, a TET2 protein, an ASXL1 protein, and/or a DNMT3A protein.

22. The cell culture of any one of claims 18-21, wherein the first HSPC comprises a marker that distinguishes the first HSPC from the second HSPC.

23. The cell culture of claim 22, wherein the marker is a fluorescent marker.

24. A method for expanding hematopoietic stem cells (HSPCs), the method comprising culturing HSPCs with endothelial cells, wherein the HSPCs comprise HSPCs having a genomic modification associated with CH.

25. The method of claim 24, wherein the HSPCs comprise:
a) a first HSPC comprising the genomic modification; and
b) a second HSPC that does not comprise the genomic modification.

26. The method of claim 24 or claim 25, wherein the endothelial cells are bone marrow endothelial cells (BMECs).

27. The method of any one of claims 24-26, wherein the first HSPC comprises a marker that distinguishes the first HSPC from the second HSPC.

28. The method of claim 27, wherein the marker is a fluorescent marker.

29. The method of any one of claims 24-28, wherein the genomic modification comprises a substitution, insertion, or deletion in a gene encoding a protein, and the genomic modification results in expression of a variant of the protein that is associated with CH.

30. The method of any one of claims 24-29, wherein the genomic modification is a substitution, insertion, or deletion in a gene encoding an IDH2 protein, a TET2 protein, an ASXL1 protein, and/or a DNMT3A protein.

31. The method of any one of claims 24-30, further comprising, prior to the culturing, infecting the HSPCs with a library of single guide RNAs (sgRNAs) comprising 10-10,000 sgRNAs.

32. The method of any one of claims 24-31, wherein the HSPCs express a programmable nuclease protein.

33. The method of claim 32, wherein the programmable nuclease protein is Cas9.

34. A method of identifying an inhibitor of clonal hematopoiesis (CH), the method comprising:
a) contacting HSPCs with a test compound, wherein the HSPCs comprise a genomic modification associated with CH;
b) culturing the contacted HSPCs with endothelial cells; and
c) measuring survival or proliferation of the cultured HSPCs contacted with the test compound, wherein a decrease in the survival or proliferation compared to a control indicates that the test compound is an inhibitor of CH.

35. The method of claim 34, wherein the genomic modification comprises a substitution, insertion, or deletion in a gene encoding a protein, and the genomic modification results in expression of a variant of the protein that is associated with CH.

36. The method of claim 34 or claim 35, wherein the genomic modification is a substitution, insertion, or deletion in a gene encoding an IDH2 protein, a TET2 protein, an ASXL1 protein, and/or a DNMT3A protein.

37. The method of any one of claims 34-36, wherein the endothelial cells are bone marrow endothelial cells (BMECs).

38. A method of inhibiting clonal hematopoiesis (CH), the method comprising:
contacting a hematopoietic stem cell (HSPC), the HSPC comprising a genomic modification associated with CH, with an agent that decreases activity of a product of a gene encoding a histone 3, lysine 9 (H3K9) demethylase.

39. The method of claim 38, wherein the genomic modification comprises a substitution, insertion, or deletion in a gene encoding a protein, and the genomic modification results in expression of a variant of the protein that is associated with CH.

40. The method of claim 38 or claim 39, wherein the genomic modification is a substitution, insertion, or deletion in a gene encoding an IDH2 protein, a TET2 protein, an ASXL1 protein, and/or a DNMT3A protein.

41. The method of any one of claims 38-40, wherein the H3K9 demethylase is KDM3B and/or JMJD1C.

42. The method of any one of claims 38-41, wherein the agent is a genome-editing agent that targets the gene encoding the H3K9 demethylase.

43. The method of any one of claims 38-42, wherein the product of the gene encoding the H3K9 demethylase is a protein.

44. The method of claim 43, wherein the agent is an antisense oligonucleotide that targets an mRNA encoding the protein.

45. The method of claim 43, wherein the agent is an inhibitor of the protein.

46. The method of claim 45, wherein the inhibitor of the protein is an enzymatic inhibitor that decreases H3K9 demethylase activity of the protein.

47. The method of claim 45, wherein the inhibitor of the protein is a mediator of protein degradation that mediates targeted degradation of the protein.

48. The method of any one of claims 38-47, wherein the HSPC is cultured with endothelial cells.

49. The method of claim 48, wherein the endothelial cells are bone marrow endothelial cells.
